(19) United States
(12) Patent Application Publication
(10) Pub. No.: US 2019/0389960 A1
(43) Pub. Date: Dec. 26, 2019

(54) COMPOSITIONS AND METHODS FOR C1Q TARGETED THERAPY

(71) Applicant: The Research Foundation for The State University of New York, Albany, NY (US)

(72) Inventors: Berhane Ghebrehiwet, Port Jefferson, NY (US); Ellinor Peerschke, Port Jefferson, NY (US)

(21) Appl. No.: 16/450,120

(22) Filed: Jun. 24, 2019

Related U.S. Application Data
(60) Provisional application No. 62/689,612, filed on Jun. 25, 2018.

(51) Int. Cl. C07K 16/28; A61P 35/00; A61K 45/06; A61K 9/00

(52) U.S. Cl. CPC C07K 16/2896; A61K 9/0019; A61K 45/06; A61P 35/00

(57) ABSTRACT

This application provides methods for treating cancer by administering to a patient a therapeutically effective amount of an agent that binds to membrane-bound C1q. The application further provides methods for inhibiting cell proliferation comprising contacting a cell expressing membrane-bound C1q with an agent that inhibits the interaction of soluble gC1qR with the membrane-bound C1q.

### STATEMENT OF GOVERNMENT INTEREST

This invention was made with government support under grant numbers AI060866 and AI084178 awarded by the National Institutes of Health. The government has certain rights in the invention.

### FIELD OF THE INVENTION

The invention relates to methods and compositions for treating cancer by targeting membrane-bound C1q and its cognate cell surface receptors, gC1qR and cC1qR.

### BACKGROUND

Human C1q (460 kDa) is the recognition unit of the classical pathway of complement, which circulates in plasma in association with the Ca²⁺-dependent C1r₂-C1s₂ tetramer (360 kDa) to form pentameric C1—the first component of complement. C1q is made up of 3 distinct polypeptide chains, A, B, and C, arranged to form 6 triple helical strands with three peptide chains—A, B, and C—forming a single strand. The three chains are the product of three distinct genes, which are highly clustered and aligned 5′ to 3′, in the same orientation, in the order A-C-B on a 24 kb stretch of DNA on the short arm of chromosome 1p (1p34.1-1p36.3). Each of the six trimeric globular “heads” of C1q, is made up of the globular domains from one A, one B, and one C chain, each of which in turn has its own ligand specificity capable of recognizing different molecular partners. The globular heads are linked via six collagen-like “stalks” to a fibril-like central region resulting in two unique structural and functional domains: the collagen-like region (cC1q) and the globular heads (gC1q). The two C1q domains can independently interact with a multiplicity of biological structures including pathogen-associated and cell-associated molecules. However, it is the gC1q domain that defines the versatility of the C1q molecule, with each of the individual globular head domain (ghA, ghB, ghC) capable of independently interacting with danger ligands (Kishore et al., 2002, Gaboriaud et al, 2007).

There are also two distinct cell receptors that preferentially recognize each region and hence are designated cC1qR and gC1qR. Binding of C1q to its globular heads receptor, gC1qR, induces complement activation (Lim et al., 1996). Through the activation of complement, C1q has the capability to recruit chemotactic and inflammatory molecules to the site of activation.

There is emerging evidence, which suggests that the role of complement component proteins in the context of cancer growth and survival extends beyond the traditional complement activation and complement mediated cell lysis. Both C1q and gC1qR are expressed on a wide range of cancer cells and play a significant role in their growth and progression. The receptor for the globular heads of C1q, gC1q-R, is a ubiquitous, highly anionic cellular protein of 33 kDa that was identified and characterized as a cell-surface molecule that binds the globular heads of C1q (gC1q) (Ghebrehiwet et al. 1994. J. Exp. Med. 179: 1809-1821). Known alternatively as p33, and sometimes as p32 or HABP-I (hyaluronic acid binding protein I), it is now clear that it is also, and in fact predominantly, a protein of the mitochondrial matrix. In addition, it is distributed in several other cellular compartments, including the ER, and the nucleus, in addition to the cell surface (Ghebrehiwet et al. 1994. J. Exp. Med. 179: 1809-1821; Mandi et al. 2001. Blood. 97:2342-2350; Mandi et al. 2002. Hemost. Thromb. Vase. Biol. 99:3585-3596). Binding of C1q to cells is known to induce and modulate a number of C1q-mediated cellular responses including inositol-trisphosphate (IP3) production in, expression of P-selectin on, and generation of procoagulant activity on, platelets; activation and expression of the adhesion molecules E-selectin, ICAM-1 and VCAM-1; and production of IL-6, IL-8, and monocyte chemoattractant protein-1 (MCP-I) on endothelial cells (ECs). Some of these functions have been shown by antibody inhibition to directly involve gC1q-R and/or the receptor for the collagen-like domains of cC1q-R/CR. In addition, gC1q-R in association with u-PAR and cytokeratin 1, is a high-affinity site for HK (Colman et al. 1997. J. Clin. Invest. 100:1481-1487; Hasan, et al. 1998. Proc. Natl. Acad. Sci. U.S.A. 95: 3615-3620).

Although the mechanism by which exogenous C1q exerts its anti-proliferative effect is yet to be determined, multiple pathways are predicted to be involved including induction of apoptosis through activation of p38 and caspase-3 and cell death with autophagy through accumulation of LC3-II and autophagosomes, as suggested. This is particularly true when one considers that C1q serves as a potent autocrine regulator of a plethora of cellular functions. For example, C1q can induce—through either cC1qR or gC1qR—the expression of TNF-αR, which in turn can activate the extrinsic apoptotic pathway and induce cell death through the TNF-α-TNFR-1 initiated apoptotic cell death pathway. Alternatively, as an ancestral molecule of the TNF family of proteins which has retained some of its TNF-like functions, exogenous C1q itself may interact with both gC1qR and TNFR-1 to activate the extrinsic apoptotic cell death pathway and induce downstream events leading to caspase-dependent cell death.

In addition to complement activation, gC1qR is also key in kinin generation through binding to the bradykinin precursor, high molecular weight kininogen (Ghebrehiwet et al., 2006). Bradykinin is integral to vascular permeability and has angiogenic and pro-proliferative functions. Kinin generation has been linked to tumor growth and metastasis (da Costa et al., 2014). Cell expression of gC1qR is upregulated in many malignant tumors and its role seems to be important to the growth of many tumor types (Dembitzer et al., 2012). C1q expression in the microenvironment has also been explored and is linked to both pro- and anti-tumor functions (Bulla et al., 2016; Miyamae et al., 2016).

### SUMMARY OF THE INVENTION

In one aspect, the present invention provides a method of treating cancer in a patient in need thereof comprising administering to the patient a therapeutically effective amount of an agent that binds to membrane-bound C1q.

In another aspect, the present invention provides a method for inhibiting cell proliferation comprising contacting a cell expressing membrane-bound C1q with an agent that inhibits the interaction of soluble gC1qR with the membrane-bound C1q.

In one embodiment the cancer is a solid tumor. In embodiments, the cancer is breast cancer.

In an embodiment of the invention, the agent is an antibody. In one embodiment, the agent is an antibody that binds to the A-chain of C1q. In another embodiment, the agent is an antibody that binds to the C-chain of C1q. In another embodiment, the agent is an antibody that binds to the gC1qR binding site on the A-chain of C1q. In another embodiment, the agent is a gC1qR polypeptide that blocks binding sites selected from the group consisting of HK and C1q binding sites. In another embodiment, the agent is an antibody or polypeptide that blocks binding of the cC1qR receptor to membrane bound C1q. In another embodiment, agent is an antibody that blocks the interaction between gC1qR and ghA of the C1q. In another embodiment, the agent is an antibody that blocks the interaction between gC1qR and ghC of the C1q.

In embodiments of the invention, the agent is injected directly into a tumor mass. In another embodiment, the agent is injected intravenously. In embodiments, the method further comprises administering to the patient an additional cancer therapy. In another embodiment, the additional cancer therapy is a monoclonal antibody. In another embodiment, the additional cancer therapy is a small molecule based immunotherapy. In another embodiment, the additional cancer therapy is radiotherapy, chemotherapy, hormonal therapy, immunotherapy, or toxin therapy.

In embodiments of the invention, the cancer is selected from the group consisting of breast cancer, mesothelioma, melanoma, colon cancer and prostate cancer. In another embodiment, the cancer is breast cancer.

The present invention provides a method for inhibiting cell proliferation comprising contacting a cell expressing membrane-bound C1q with an agent that inhibits the interaction of soluble gC1qR with membrane-bound C1q. In an embodiment, the agent is an antibody. In another embodiment, the antibody binds to the membrane-bound C1q. In another embodiment, the antibody binds to the A-chain of C1q. In another embodiment, the agent is an antibody that binds to the C-chain of C1q. In another embodiment, the antibody binds to the gC1qR binding site on the A-chain of C1q. In another embodiment, the antibody blocks the interaction between gC1qR and ghA of the C1q. In another embodiment, the antibody blocks the interaction between gC1qR and ghC of the C1q.

### BRIEF DESCRIPTION OF THE FIGURES

FIG. 1 shows the cell surface expression of gC1qR on SkBr3 cells. Deconvolution fluorescence microscopy was performed on SkBr3 cells grown to confluency on glass cover slips. Cells were incubated with PBS containing 0.1% BSA and 1 mg/ml human Fc fragments to block FcRs, followed by incubation with immunoaffinity purified rabbit anti-gC1qR peptides (A) recognizing either residues in the pre-pro sequence (aa50-63) or in the mature protein (aa144-155). (B) Bound Abs were probed with Alexa Fluor 488-conjugated goat anti rabbit Abs (GāR). This experiment is a representative of three identical experiments. Control slide was stained with Alexa Fluor 488 anti-rabbit Abs. Original magnification ×68.

FIG. 2 shows co-localization of membrane bound gC1qR and cC1qR on the SkBr3 cell surface. SkBr3 cells grown on glass cover slips were incubated with PBS containing 0.1% BSA and 1 mg/ml Fc fragments to block FcRs. The cells were then probed with mAb 74.5.2 anti-gC1qR and rabbit pAb anti-cC1qR recognizing residues 141-151. Bound Abs were then probed with Alexa Fluor 488-anti-rabbit Abs or Alexa Fluor 594 anti-mouse Abs. Co-localization of gC1qR and cC1qR is shown in the merged picture. Control staining with rabbit non-immune IgG for both experiments showed no fluorescence and was not included.

FIG. 3 shows cell surface expression of C1q by SkBr3 cells. (A) Flow cytometry analysis was performed on SkBr3 cells grown to confluency and detached using 10 mM EDTA solution. Cells were then blocked with human Fc fragments for 30 min on ice and subsequently probed with either 10 μg of non-immune rabbit IgG, rabbit anti-C1q, or rabbit anti-gC1qR for 30 min on ice. Then, Alexa flour 488 conjugated mouse anti-rabbit Ab was added and incubated for 30 min on ice. The cells were stained with propidium iodide to assess viability. (B) Immunofluorescence studies on SkBr3 cells grown in chamber slides to 90% confluency were performed to determine C1q expression. Monoclonal anti-C1q and goat anti-C1q were used as well as the isotype matched non-immune IgG controls for 30 min at room temp. After incubation with Alexa fluor 488-conjugated donkey anti-mouse or rabbit anti-goat antibodies (30 min), DAPI stain was applied to visualize the nucleus. The images represent multiple locations for each condition.

FIG. 4 shows C1q expression on SkBr3 cells. Western blot analysis on SkBr3 cell lysates was done to detect expression of C1q. Cell lysates as well as purified C1q (20 ng) were first run on a 10% polyacrylamide gel and then transferred onto nitrocellulose membranes. The membranes were then blocked with 2% BSA in TBST and incubated overnight at 4° C. with either rabbit non-immune IgG, or antibodies recognizing C1q, the A-chain of C1q or the globular head domain of the A-chain (ghA). The proteins were then detected using HRP-conjugated rabbit antibody and a chemiluminescence substrate and exposed on film. The figure is a representative of three such experiments.

FIG. 5 shows the effect of anti-C1qR antibody. SkBr3 cells were seeded in 6 well plates at 1×10⁵ cells/ml in the presence or absence of either (A) 10 μg/ml mAb 60.11 recognizing the C1q site on gC1qR or (B) 10 μg/ml affinity-purified rabbit anti-cC1qR recognizing the putative C1q binding domain on cC1qR. After 96 hr, cells were counted in a hemocytometer in the presence of 10 μl/ml trypan blue for cell viability. Results show cell proliferation levels represented as the mean of four assays run in duplicates. Values represent mean±SD of duplicate samples with significance represented by (*p<0.05) and *** (p<0.005) when compared to control samples.

FIG. 6 shows that the A-chain of C1q is important for cell survival. Proliferation assays were conducted using SkBr3 cells seeded at 1×10⁵ cells/ml in the presence or absence of rabbit anti-C1q (10 pg/ml), anti C1qA (10 pg/ml), or rabbit anti-ghA. Cells were then incubated for 96 hr, after which they were counted in a hemocytometer in the presence of trypan blue. Control wells were either untreated or supplemented with isotype-matched non-immune rabbit IgG (NIRG). Results for NIRG supplemented cells were no different from untreated cells (control) and are not included here. Results are representative of four different experiments run in duplicates. Significance is represented by (** p<0.01) and *** (p<0.005) when compared to control using student's t-test.

FIG. 7 shows the anti-proliferative effect of C1q and its globular head modules on SkBr3 cells. SkBr3 cells were seeded in 6 well plates at 1×10⁵ cells/ml in the presence or absence of either 10 pg/ml (A) C1q, (B) ghA, ghB, or ghC. Each plate was counted after 96 hr using a hemocytometer in the presence of trypan blue. Results show cell proliferation levels represented as the mean of four assays run in duplicates. Values represent mean±SD of duplicate samples with significance (* p<0.05) and *** (p<0.005) when compared to control using student's t-test.

FIG. 8 shows the SkBr3 cell secreted gC1qR reverses the anti-proliferative response of C1q. (A) Antigen-capture-ELISA on SkBr3 cell supernatants grown in serum-free RPMI for 24, 48, or 72 hrs (n=4) was performed using microtiter plate wells coated with 100 μl of immunoaffinity purified (10 pg/ml) rabbit anti-gC1qR peptide 144-155 (1 hr, 37° C.). After blocking with 1% BSA (1 hr, 37° C.), 100 μl of either fresh medium or culture supernatants was added and further incubated (1 hr, 37° C.). The captured gC1qR was then detected by sequential incubation (1 hr, 37° C.) with biotinylated mAb 60.11 (2 pg/ml), alkaline phosphatase conjugated-Neutravidin and PNPP as described in the Examples. (B) Cell proliferation assay was conducted to assess if gC1qR released by SkBr3 cells, could neutralize the anti-proliferative effect of exogenously added C1q. To this end, SkBr3 cells were first seeded in duplicate wells at 1×10⁵ cells/ml and grown for 48 hr to allow optimal gC1qR secretion (n=2). Then, the culture medium was removed from half of the wells and replaced with fresh medium, while the other half of the wells remained in the gC1qR-rich “old” medium. Next, C1q was added to both wells and incubated for 96 hr after which, the cells in each well were counted and viability assessed using the trypan blue exclusion assay. Significance is represented by (** p<0.01) and *** (p<0.005) when compared to control using student's t-test.

FIG. 9 shows soluble gC1qR is an autocrine signal of cell proliferation. (A) Proliferation assay was or 10 pg/ml (n=2). After 96 hr, cells were trypsinized and done in which SkBr3 cells at 10⁵/ml were co-cultured with gC1qR at 5 pg/ml counted to determine cell number and viability. (B) Cells were visualized and counted at different time points to determine the time-dependent effect of gC1qR on cell number and viability. Values represent mean±SD of duplicate samples with significance of (* p<0.05), (** p<0.01), and (***p<0.005), when compared to untreated controls.

FIG. 10 shows an adhesion assay on C1q- and gC1qR-coated plates. Microtiter plates (12-well) were coated (overnight at 20° C. under sterile conditions) with either 20 μg/ml of C1q or gC1qR in carbonate buffer, pH 9.6 (15 mM Na₂CO₃ and 35 mM NaHCO₃). Excess buffer was removed and washed with sterile PBS before the addition of 1×10⁵ cells/well in 1 ml of RPMI medium. (A) A light microscope was used to take 4× images of the wells every 24 hours for 96 hours. Buffer coated wells were used as control and did not show any significant difference to cells in culture medium-coated wells. The images are representative of 3 experiments run in triplicates. (B) Cell supernatants were collected after 96 hr, and the remaining adherent cells were trypsinized after which supernatants and trypsinized cells were combined, stained with trypan blue and viability established. Students t-tests were applied to determine significance (p<0.05*, p<0.01**).

FIG. 11 shows a schematic representation depicting the current understanding of roles of membrane bound and soluble forms of C1q and gC1qR. While C1q and gC1qR are membrane-associated proteins and both function to induce pro-apoptotic signals by recruiting suitable signaling partners [(e.g. CD44, or receptor tyrosine kinases (RTKs)], the secreted or released forms have different functions. Whereas soluble C1q binds to the cell via gC1qR and triggers an anti-proliferative signal, soluble gC1qR induces a pro-proliferative signal ostensibly through the globular heads of membrane C1q. Importantly, gC1qR released into the tumor cell microenvironment (sgC1qR) acts as a pluripotent molecular orchestrator triggering activation of both the complement and kinin systems and in the process releasing reactive products (C1q, HKa, BK, IL-1β), which together provide a pro-proliferative and angiogenic microenvironment. In addition, metastasis to distal organs is facilitated through generation of BK.

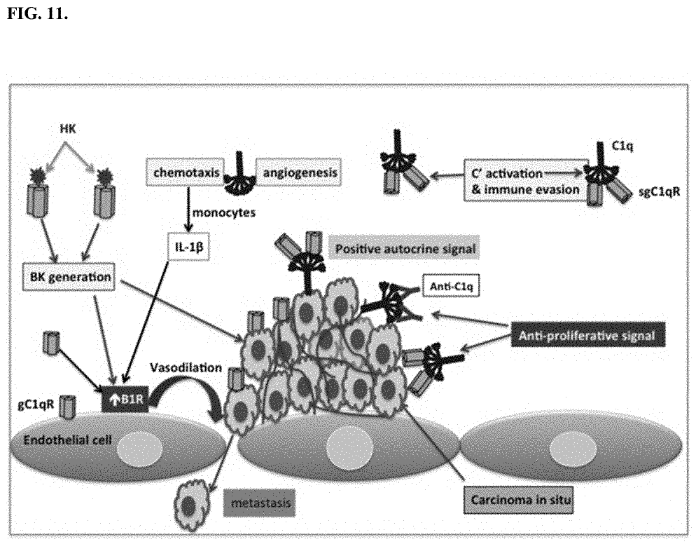

FIG. 12 shows an ELISA assay showing interaction of ghA mutants with gC1qR. Microtitre wells were coated with 1 μg/well of gC1qR in carbonate buffer and double diluted 2 wells down to give a concentration of 0.5, 0.25 μg/well. The plate was incubated at +4 overnight. The next morning, contents were discarded and wells were blocked for 2 hours with 2% BSA at 37 degrees. Following washing with PBS+0.05% Tween, 2.5 μg/well of ghA wild type, R162A, R162E and MBP was added and the plate was incubated for 1.5 hours at 37 degrees and 1.5 hours at +4. Wells were washed an anti-MBP (1/5000) was added and incubated for 1 hour. Bound protein was detected using IgG-HRP and color was developed using OPD buffer. The plate was read at a wavelength of 450 nm.

FIG. 13 shows the antiproliferative effect of C1q and its gh modules on SkBr3 cells. SkBr3 cells were seeded in 6-well plates at 1×10⁵ cells/ml in the presence of either (A) C1q, (B) purified ghA, ghB, or ghC. After 96 hr, viability was assessed using the trypan blue exclusion assay and viable and non-viable cells counted in a hemocytometer. Results show cell proliferation levels and are represented as the mean of four assays run in duplicates. Significance is represented by (*p<0.05) and ***(p<0.005) when compared to control using student's t-test.

FIG. 14 shows a comparison of the anti-proliferative effect of C1q and its gh modules on SkBr3 cells. SkBr3 cells were seeded in 6-well plates at 1×10⁵ cells/ml with or without C1q (A), ghA, ghB, or ghC (B), or C1q or ghA (C) at 10 μg/ml. Cells were counted either (A, B) after 96 hrs or (C) after 48, 72 and 96 hrs using separate plates each time. Cell viability was assessed using the trypan blue exclusion assay. Cell proliferation levels represented as the mean of 4 separate assays run in duplicates are depicted. Significance is represented by (*p<0.05) and ***(p<0.005) when compared to untreated control using the student's t-test.

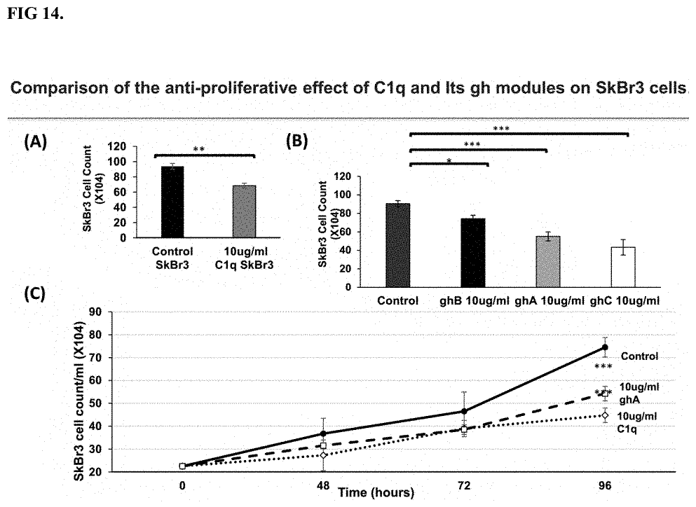

FIG. 15 shows that unlike C1q, soluble gC1qR enhances SkBr3 cell proliferation even in the absence of enriched medium. SkBr3 cells were seeded at 10⁵/ml in 6 well plates (n=2) in the presence or absence of 6 μg/ml or 10 μg/ml gC1qR (A). After 96 hrs, cells were trypsinized and counted with a hemocytometer using a trypan blue exclusion assay to determine cell number and viability. (B) cells were grown for 8 days without replenishment of medium to allow for depletion of nutrients. Cells were counted as described above and % viability was determined for all conditions. Student's t-tests were done for all experiments and the significance of results is represented by (*p<0.05), (**p<0.01), and (***p<0.005).

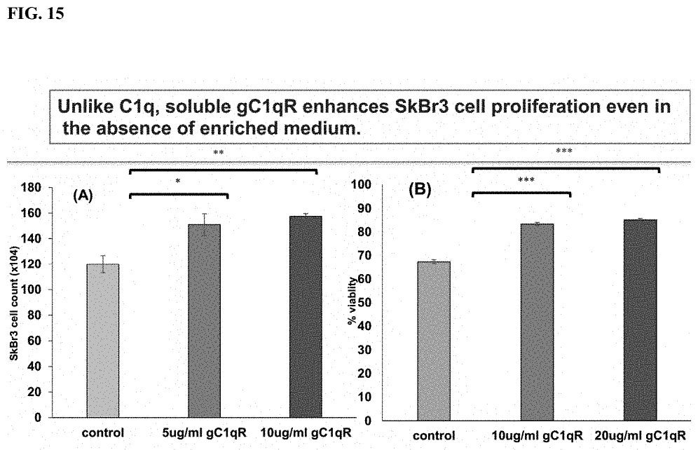

FIG. 16 shows the structural organization of the C1q molecule. Intact C1q is made up of 6A, 6B and 6C chains (A). The chains are arranged to form 6 individual strands and each strand is made up of a disulfide bonded A-B dimer non-covalently associated with a C-chain (B). Two strands are disulfide-bonded through adjacent C-chains to form a doublet and three doublets are non-covalently linked to form the intact hexameric C1q (C).

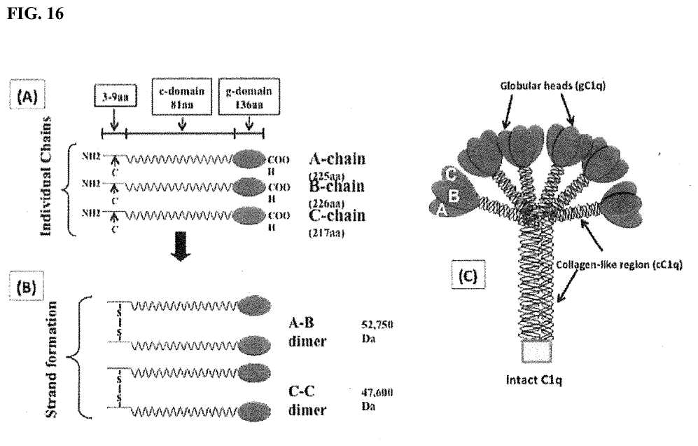

FIG. 17 shows the homotrimeric structure of gC1qR can potentially bind three globular heads of C1q and conversely C1q can bind potentially two molecules of gC1qR. This multi-site binding interaction explains the high affinity interaction between the two molecules both in fluid phase and on the cell surface.

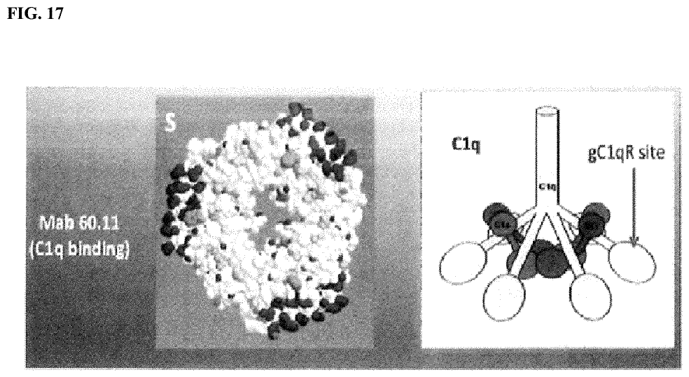

FIG. 18 shows the functional domains of the C1qA chain. The intact C1q molecule is anchored to the cell membrane by a leader peptide in the A chain at (−17)-(−1). In addition, the versatility of the A-chain is depicted here with the MHC class II binding domain as well as a major non-IgG binding region. The major gC1qR binding domain spanning residues 155-164 and another at unexpected collagen domain spanning residues 14-26 and another at 76-92 are also shown. (Adapted from Tridner P. K. et al; Behring Inst. Mitt. 1993, 180-188).

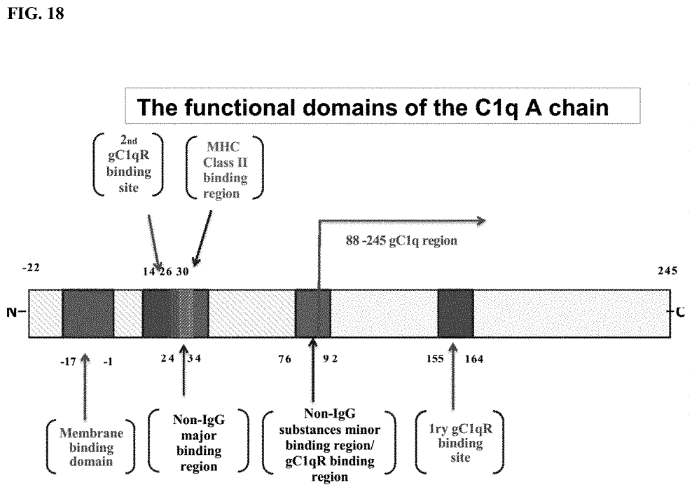

### DETAILED DESCRIPTION OF THE INVENTION

One aspect of the present invention provides a method of treating cancer in a patient in need thereof comprising administering to the patient a therapeutically effective amount of an agent that is capable of binding to membrane-bound C1q. The present invention also provides a method for inhibiting cell proliferation comprising contacting a cell expressing membrane-bound C1q with an agent that inhibits the interaction of soluble gC1qR with the membrane-bound C1q.

Both C1q and gC1qR are expressed on a wide range of cancer cells and play a significant role in their growth and progression. Using several tumor-derived cell lines such as Raji, Daudi, Wi12WT and U937 cells it has been shown that co-culture of these cells with purified C1q inhibits their proliferation. This proliferative response in turn was hypothesized to be mediated via the interaction of exogenous C1q with cell surface C1qR. Blockade of gC1qR with either C1q or monoclonal antibody to gC1qR (mAb 60.11), which recognizes the C1q site on gC1qR inhibited cell proliferation. Furthermore, interaction between C1q and gC1qR occurs predominantly via a site located on the globular head region of the A-chain (ghA) and to a lesser extent via the ghC chain. Addition of the individual recombinant gh domains i.e. ghA, ghB and ghC resulted in hierarchical inhibition of cell proliferation ghA=ghC>ghB.

While elevated expression of both molecules on the cell surface is pro-proliferative, in that cells divide and grow well, addition of either fluid phase C1q or gC1qR to cell cultures have diametrically opposed functions. While addition of C1q completely inhibits cell proliferation by binding to cell surface gC1qR, addition of gC1qR enhances proliferation by binding to one of several candidate cell surface signaling molecules including cell surface C1q itself.

Embodiments of the invention described herein provide compositions that target both membrane bound C1q and gC1qR either as single targets or dual targets. Use of an antibody that recognizes the C1q interaction site on gC1qR (residues 74-95) (for example mAb 60.11) as potential therapy for the treatment of cancer especially at the early stages when a tumor grows as a tumor cell cluster where the cancer is still restricted in situ and has not yet metastasized. The antibody may be injected directly into the tumor mass thus obviating the systemic injection, which could otherwise affect off target cells and interfere with their physiologic functions. This is particularly true for gC1qR, which is ubiquitously distributed in tissues and cells throughout the body. C1q on the other hand is distributed in plasma and in specific cell types such as epithelial cells, fibroblasts, and antigen presenting cells such as monocytes and dendritic cells.

According to a further embodiment, a monoclonal antibody may be generated to either the A-chain of C1q or to a domain corresponding to the gC1qR site on the A chain, which comprises of residues 155-164. According to a further embodiment, a monoclonal antibody may be generated to the C-chain of C1q.

Inhibiting Tumor Cell Proliferation by Targeting Membrane-Bound C1q

Methods and use of the compositions of the present invention are based on the recognition that membrane bound C1q is involved in the proliferation of tumor cells. Therefore, agents that target membrane bound C1q would be considered therapeutic agents. Agents may include an antibody, a polypeptide, a nucleic acid, a small molecule, or any other suitable antagonist of membrane bound C1q and its cellular proliferation promoting effects. The agents are sufficient to block binding, completely or partially, of gC1qR at a therapeutically acceptable dose.

Treatment and Prophylaxis

The terms ameliorate and treat are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease and/or improve the symptoms associated with the disease. They should not be taken to imply that a subject is treated to a total recovery.

The invention provides for treating cancer in a patient in need thereof comprising administering to the patient a therapeutically effective amount of an agent that is capable of binding to membrane-bound C1q. In embodiments of the invention, the agent binds to membrane bound C1q on cancer cells. In further embodiments, the agent specifically blocks the interaction of the membrane bound C1q with soluble gC1qR.

In embodiments of the invention, the method comprises treatment primary or metastasized tumors of ovary, breast, brain, head and neck, liver, lung, prostate, kidney, colon, pancreas, thyroid, urinary bladder, abdominal cavity, thoracic cavity and skin. In an embodiment, the method comprises treating primary or metastasized tumors of breast cancer, mesothelioma, melanoma, colon cancer and prostate cancer. In embodiments, the method comprises treatment of adenocarcinomas including, for example, adenocarcinoma of the lung, breast, colon, pancreas, and ovary.

As used herein, the term “therapeutically effective amount” refers to the quantity or amount of a C1q binding agent (e.g., an antibody or pharmaceutical composition provided herein) which is sufficient to reduce, diminish, alleviate, and/or ameliorate the severity and/or duration of a cancer or a symptom related thereto. This term also encompasses an amount necessary for the reduction or amelioration of the advancement or progression of a cancer; the reduction or amelioration of the recurrence, development, or onset of a cancer; and/or the improvement or enhancement of the prophylactic or therapeutic effect(s) of another cancer therapy (e.g., a therapy other than administration of an antibody that is capable of binding to membrane-bound C1q provided herein). In some embodiments, the effective amount of an antibody provided herein is from about or equal to 0.1 mg/kg (mg of antibody per kg weight of the subject) to about or equal to 100 mg/kg. In certain embodiments, an effective amount of an antibody provided therein is about or equal to 0.1 mg/kg, about or equal to 0.5 mg/kg, about or equal to 1 mg/kg, about or equal to 3 mg/kg, about or equal to 5 mg/kg, about or equal to 10 mg/kg, about or equal to 15 mg/kg, about or equal to 20 mg/kg, about or equal to 25 mg/kg, about or equal to 30 mg/kg, about or equal to 35 mg/kg, about or equal to 40 mg/kg, about or equal to 45 mg/kg, about or equal to 50 mg/kg, about or equal to 60 mg/kg, about or equal to 70 mg/kg, 80 mg/kg, 90 mg/kg, or 100 mg/kg. These amounts are meant to include amounts and ranges therein. In some embodiments, “effective amount” also refers to the amount of an antibody provided herein to achieve a specified result (e.g., binding to membrane bound C1q). In embodiments, the C1q binding agent is administered directly to a tumor. When administered directly to the tumor, about 1 mg to about 5 mg of the C1q binding agent may administered directly to the tumor, depending on the size of the tumor.

Administration

As used herein, the terms “administer” or “administering” refer to the addition of a substance to the body of a subject, including for example local (as opposed to systemic) administration. In particular embodiments, the disclosed agents may be administered by any appropriate route, including but not limited to intravenous injection, intralymphatic injection, parenteral injection, peritoneal injection, subcutaneous injection, intracutaneous injection, intratumoral injection, peritumoral injection, intradermal injection (such as into the areola), injection into the lymphatic system, injection into a surgical field, and subdermal injection. Other means of administration can be used, including oral, buccal, sublingual, and rectal administration and by intravenous or intraperitoneal infusion. Agents may be prepared for administration by conventional pharmacological means, such as by adding excipients, fillers or diluents, buffers, stabilizers, flavorings, solubilizers, antibacterial agents, antifungal agents, isotonic agents, and the like.

The agent may be administered more than once, and/or administered until the patient enters remission. The method may further comprise administering to the patient one or more additional cancer therapies, such as radiotherapy, chemotherapy, hormonal therapy, immunotherapy, surgery and/or toxin therapy.

Thus, some embodiments relate to a pharmaceutical composition for the treatment of abnormal cell growth or proliferation in a patient, which comprises an amount of a C1q binding agent in combination with one or more additional anti-cancer agents, for example, anti-angiogenesis agents and signal transduction inhibitors, wherein the amounts of the active agent and the combination anti-cancer agents when taken as a whole is therapeutically effective for treating the abnormal cell proliferation.

Some embodiments relate to a method for the treatment of breast cancer in a human in need of such treatment, comprising administering to the human an amount of a C1q binding agent, in combination with one or more anticancer agents selected from the group consisting of trastuzumab, tamoxifen, docetaxel, paclitaxel, capecitabine, gemcitabine, vinorelbine, exemestane, letrozole cetuximab and anastrozole.

When combined with other anti-cancer agents, the compositions of the present invention comprising a C1q binding agent can be administered concurrently (in the same or separate compositions) or sequentially.

Antibodies

As described herein, blocking membrane-bound C1q with an antibody targeting the C1qR binding site has an antiproliferative effect. The present studies examined more closely the roles played by these molecules employing the SkBr3 cancer cell as a model for breast cancer. The present invention provides compositions and methods directed to monoclonal antibody-based therapy by targeting C1q and its cognate cell surface receptors, cC1qR and gC1qR.

The complete nucleotide and amino acid sequences of human C1QA complement C1q A chain can be found under GenBank accession number NG_007282.1. The complete nucleotide and amino acid sequences of C1QB complement C1q B chain can be found under GenBank accession number NG_007283.1. The complete nucleotide and amino acid sequences of human C1QC complement C1q C chain can be found under GenBank accession number NG_007565.1. The complete nucleotide and amino acid sequences of human gC1qR can be found under GenBank accession number NC_000017.11. The complete nucleotide and amino acid sequences of human cC1qR can be found under GenBank accession number NG_029662.1. Each of these nucleotide and amino acid sequences are incorporated herein by reference.

Antibodies of the present invention bind to membrane bound antibodies of the invention include those consisting of heavy and light chains in their natural configuration, and functional fragments or modifications thereof. The antibodies of the present invention may be monoclonal or polyclonal raised in animals, but can also be humanized, super-humanized, chimeric antibodies, or human antibodies. The term monoclonal antibody as used herein refers to an antibody obtained from a substantially homogeneous population of antibodies. The antibodies within the population are identical, except for possible mutations occurring in a small subset of the antibodies. As used herein, monoclonal antibodies include chimeric antibodies, in which constant regions are obtained from one source and variable regions from a different source, humanized antibodies in which all but the complementarity determining regions (CDRs) are human, and “superhuman” antibodies in which human CDRs are incorporated into human variable domains. Antibodies that can be used according to the invention include complete immunoglobulins, antigen binding fragments of immunoglobulins, as well as antigen binding proteins that comprise antigen-binding domains of immunoglobulins. Antigen binding fragments of immunoglobulins include, for example, Fab, Fab′, and F(ab′)₂. Other antibody formats have been developed which retain binding specificity, but have other characteristics that may be desirable, including for example, bi-specificity, multi-valence (more than two binding sites), and compact size (e.g., binding domains alone).

Naturally occurring antibodies typically have two identical heavy chains and two identical light chains, with each light chain covalently linked to a heavy chain by an interchain disulfide bond. Multiple disulfide bonds further link the two heavy chains to one another. Individual chains can fold into domains having similar sizes (110-125 amino acids) and structures, but different functions. The light chain can comprise one variable domain (VL) and/or one constant domain (CL). The heavy chain can also comprise one variable domain (VH) and/or, depending on the class or isotype of antibody, three or four constant domains (C_{R}1, C_{R}2, C_{R}3 and C_{R}4). In humans, the isotypes are IgA, IgD, IgE, IgG, and IgM, with IgA and IgG further subdivided into subclasses or subtypes (IgA1-2 and IgG1-4).

Generally, the variable domains show considerable amino acid sequence variability from one antibody to the next, particularly at the location of the antigen-binding site. Three regions, called hypervariable or complementarity-determining regions (CDRs), are found in each of VL and VH, which are supported by less variable regions called framework variable regions.

The portion of an antibody consisting of VL and VH domains is designated Fv (fragment variable) and constitutes the antigen-binding site. Single chain Fv (scFv) is an antibody fragment containing a VL domain and a VH domain on one polypeptide chain, wherein the N terminus of one domain and the C terminus of the other domain are joined by a flexible linker (see, e.g., U.S. Pat. No. 4,946,778 (Ladner et al); WO 88/09344, (Huston et al). WO 92/01047 (McCafferty et al) describes the display of scFv fragments on the surface of soluble recombinant genetic display packages, such as bacteriophage.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first polypeptide linker, can be covalently linked by at least one or more peptide linker to form a multivalent single chain antibodies, which can be monospecific or multispecific. Each chain of a multivalent single chain antibody includes a variable light chain fragment and a variable heavy chain fragment and is linked by a polypeptide linker to at least one other chain. The polypeptide linker is composed of at least fifteen amino acid residues. The maximum number of amino acid residues is about one hundred.

Two or more single chain antibodies can be constructed so as to associate into complexes having more than one antigen-binding site. For example, two chains can be combined to form a diabody (i.e., a bivalent dimer). Diabodies have two binding sites and can be monospecific or bispecific. Each chain of the diabody includes a VH domain joined to a VL domain with a linker short enough to prevent pairing between domains on the same chain. Thus, complementary domains on different chains pair with one another to recreate the two antigen-binding sites. Similarly, three chains can be combined to form a triabody. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Fab (Fragment, antigen binding) refers to the fragment of an antibody consisting of VL, CL, VH, and CHl domains. Such fragments can be generated by papain digestion of an antibody, or expressed from nucleic acids encoding those domains. F(ab′)2 refers to the fragment of an antibody obtained by digestion with pepsin. F(ab′)2 antibody fragments also contain VL, CL, VH, and CHl domains, as well as a heavy chain hinge region through which dimers are formed. Such fragments can also be generated using recombinant DNA techniques.

Fc (Fragment crystallization) is the designation for the portion or fragment of an antibody that comprises paired heavy chain constant domains. In an IgG antibody, for example, the Fc comprises C_{R}2 and C_{R}3 domains. The Fc of an IgA or an IgM antibody further comprises a C_{H}4 domain. The Fc is associated with Fc receptor binding, activation of complement-mediated cytotoxicity, and antibody-dependent cellular-cytoxicity (ADCC). For antibodies such as IgA and IgM, which are complexes of multiple IgG like proteins, antibody formation requires Fc constant domains.

Specificity of antibodies, or fragments thereof, can be determined based on affinity and/or avidity. Affinity, represented by the equilibrium constant for the dissociation of an antigen with an antibody (Kd), measures the binding strength between an antigenic determinant and an antibody-binding site. Avidity is the measure of the strength of binding between an antibody with its antigen. Avidity is related to both the affinity between an epitope with its antigen-binding site on the antibody, and the valence of the antibody, which refers to the number of antigen binding sites of a particular epitope. Antibodies typically bind with a dissociation constant (Kd) of 10^{˜5} to 10^{˜π} liters/mol. Any Kd less than 10^{˜4} liters/mol is generally considered to indicate nonspecific binding. The lesser the value of the Kd, the stronger the binding strength between an antigenic determinant and the antibody-binding site.

As used herein, “antibodies” and “antibody fragments” includes modifications that retain specificity for a specific antigen. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). The antibodies can be modified by conjugation to detectable reporter moieties. Also included are antibodies with alterations that affect non-binding characteristics such as half-life (e.g., pegylation).

Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50:6600-6607 (1990) for the conjugation of doxorubicin and those described by Arnon et al., Adv. Exp. Med. Biol. 303, 79-90 (1991) and by Kiseleva et al., MoI. Biol. (USSR) 25:508-514 (1991) for the conjugation of platinum compounds.

Antibodies and antibody fragments of the present invention further include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al, J. Mol. Biol, 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol, 226: 889-896 (1992)). For example, phage display vectors containing heavy and light chain variable region genes can be propagated in mutator strains of E. coli (see, e.g., Low et al., J. Mol. Biol, 250: 359-368 (1996)). These methods of mutagenesis are illustrative of the many methods known to one of skill in the art.

Each domain of the antibodies of this invention can be a complete immunoglobulin domain (e.g., a heavy or light chain variable or constant domain), or it can be a functional equivalent or a mutant or derivative of a naturally-occurring domain, or a synthetic domain constructed, for example, in vitro using a technique such as one described in WO 93/11236 (Griffiths et al.). For instance, it is possible to join together domains corresponding to antibody variable domains, which are missing at least one amino acid. The important characterizing feature of the antibodies is the presence of an antigen binding site. The terms variable heavy and light chain fragment should not be construed to exclude variants that do not have a material effect on specificity.

Preparation of Antibodies

Antibodies of the present invention can be obtained by any technique, for example, from naturally occurring antibodies, or Fab or scFv phage display libraries. It is understood that, to make a single domain antibody from an antibody comprising a VH and a VL domain, certain amino acid substitutions outside the CDRs can be desired to enhance binding, expression or solubility. For example, it can be desirable to modify amino acid residues that would otherwise be buried in the VH-VL interface

Antibodies of the invention can be obtained by standard hybridoma technology (Harlow & Lane, ed., Antibodies: A Laboratory Manual, Cold Spring Harbor, 211-213 (1988), which is incorporated by reference herein). Human monoclonal antibodies can be made, for example, by priming of B cells and fusion to create a heterohybrid (Boerner et al., 1991, J. Immunology., 147(1):86-95), or by EBV transformation of human B cells (Traggiai et al., 2004, Nat. Med. 10:871-5), and the binding characteristics of such antibodies and be modified or improved by known methods (See, Li et al., 2006, Proc. Natl. Acad. Sci. USA 103:3557-62). Human monoclonal antibodies can also be obtained from transgenic mice that produce human immunoglobulin gamma heavy and kappa light chains. In one embodiment, a substantial portion of the human antibody producing genome is inserted into the genome of the mouse, which is rendered deficient in the production of endogenous murine antibodies. Such mice may be immunized subcutaneously with part or all of target molecule in complete Freund's adjuvant. Human antibodies can also be developed using phage display techniques (See, e.g., Hoogenboom et al., 1991, J. MoI. Biol. 227:381; Marks et al, 1991, J. MoI. Bio. 222:581).

Antibodies to C1q described herein may be produced using any methods described herein or known in the art. Monoclonal antibodies (e.g., human antibodies) of the invention can be produced using known techniques, such as the standard somatic cell hybridization technique described by Kohler and Milstein, Nature 256: 495 (1975). Other techniques for producing monoclonal antibodies to C1q can also be employed, e.g., viral or oncogenic transformation of B lymphocytes and phage display techniques using libraries of human antibody genes.

One method for generating hybridomas which produce monoclonal antibodies of the invention is the murine system. Hybridoma production in the mouse is well known in the art, including immunization protocols and techniques for isolating and fusing immunized splenocytes. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating an anti-C1q monoclonal antibody. Moreover, the ordinarily skilled artisan will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

Additionally, recombinant anti-C1q antibodies, such as chimeric, composite, and humanized monoclonal antibodies, which can be made using standard recombinant DNA techniques, can be generated. Such chimeric, composite, and humanized monoclonal antibodies can be produced by recombinant DNA techniques known in the art, for example using methods described in Cabilly et al. U.S. Pat. No. 4,816,567; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Pharmaceutical Compositions

A therapeutically effective amount of the pharmaceutical composition of the present invention is sufficient to treat and/or prevent cancer. The dosage of active agent(s) may vary, depending on the reason for use, the individual subject, and the mode of administration. The dosage may be adjusted based on the subject's weight, the age and health of the subject, and tolerance for the compound or composition.

In another aspect, the present invention provides pharmaceutically acceptable compositions which comprise a therapeutically effective amount of a C1q binding agent effective at treating or preventing cancer, formulated together with one or more pharmaceutically acceptable excipients. The active agent and excipient(s) may be formulated into compositions and dosage forms according to methods known in the art. The pharmaceutical compositions of the present invention may be specially formulated for administration in solid or liquid form, including those adapted for parenteral administration, for example, by subcutaneous, intratumoral, intramuscular or intravenous injection as, for example, a sterile solution or suspension. Other modes of administration that may be employed depending on the nature of the C1q binding agent and the particular disease to be treated include: (1) oral administration, for example, tablets, capsules, powders, granules, pastes for application to the tongue, aqueous or non-aqueous solutions or suspensions, drenches, or syrups; (2) topical application, for example, as a cream, ointment or spray applied to the skin, lungs, or mucous membranes; or (3) intravaginally or intrarectally, for example, as a pessary, cream or foam; (4) sublingually or buccally; (5) ocularly; (6) transdermally; or (7) nasally.

Therapeutic compositions comprising antibodies that bind to C1q may formulated with one or more pharmaceutically-acceptable excipients including, for example, a bulking agent, salt, surfactant and/or preservative. A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68 etc).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

The phrase “pharmaceutically-acceptable excipient” as used herein refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the patient. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

The composition of the invention may be administered alone or may be administered in combination with one or more of other therapeutic agents. In one embodiment the composition of the present invention is administered in combination with one or more other anti-cancer agents.

The term “patient” as used herein refers to any organism in need of treatment, or requiring preventative therapy for cancer with the methods and compositions of the invention. The patient may be livestock, such as cattle, swine, sheep, poultry, and horses, or pets, such as dogs and cats. The patient may also be a human.

All references referred to herein are incorporated in their entirety.

### EXAMPLES

Chemicals and General Reagents

The following reagents and chemicals were purchased or obtained from the sources indicated: Dulbecco's PBS (D-PBS) without calcium and magnesium (Mediatech Inc, Manassas, Va.); RPMI 1640, 100× Penicillin/Streptomycin, and trypsin/EDTA (GIBCO-Invitrogen, Grand Island, N.Y.); heat inactivated fetal bovine serum (FBS) (Hyclone, Logan, Utah); p-nitrophenyl phosphate (pNPP) (Pierce, Rockford, Ill.); Immu-Mount (Thermo Fisher, Waltham, Mass.). Alexa 488- or Alexa 594-Streptavidin, Alexa 488- or Alexa 594-F(ab′)2, goat anti mouse or anti rabbit; FITC-conjugated goat anti-mouse IgG F(ab′)2 or sheep anti-rabbit IgG F(ab′)2 (Invitrogen, Carlsbad, Calif.); alkaline phosphatase (AP)-conjugated goat anti-rabbit IgG (Pierce), Mini PROTEAN precast TGX gels (Biorad CA), Pierce Cell Surface Protein isolation kit (Thermo Fisher, Waltham Mass.).

Expression of Various Versions of Recombinant gC1qR Proteins

The strategy for the construction of plasmids containing the full-length gC1qR was described in detail in Ghebrehiwet, B. et al., J Exp Med 179:1809-1821 (1994); Ghebrehiwet, B., et al. Immunobiology 205:421-432 (2002); Lim, B., et al. J Biol Chem 271:26739-26744 (1996), each incorporated herein by reference.

The recombinant globular head proteins, ghA, ghB, ghC, were expressed as a fusion with MBP in E. coli BL21 strain, and the recombinant proteins purified as described in Kojouharova, M. S., et al. J Immunol 172: 4351-4358 (2004) and Kishore, U., et al. J Immunol 171: 812-820 (2003), each incorporated herein by reference.

Proteins and Antibodies

The purified proteins used in these studies were obtained from the following sources. Monoclonal as well as polyclonal antibodies to recombinant human gC1qR, to cC1qR have been described previously (Ghebrehiwet, B., et al. Immunobiology 205: 421-432 (2002); Ghebrehiwet, B., et al. Hybridoma. 15: 333-342 (2009) incorporated herein by reference). In addition, immunoaffinity purified antibodies were made to selected synthetic peptides from gC1qR. Rabbit anti-C1q was made and purified in our laboratory; goat anti-C1q and monoclonal anti-C1q as well as purified C1q were purchased from Quidel (San Diego, Calif.). Rabbit anti C1q-A chain was purchased from Thermofisher Scientific LLC.

Cultured Cells

The SkBr3 cell line was purchased from ATCC and grown in RPMI 1640 containing 10% heat inactivated fetal bovine serum and 100 U/ml penicillin and 100 pg/ml streptomycin (GIBCO-Invitrogen, Grand Island N.Y.) and maintained in a humidified air consisting of 5% CO₂ and 95% air. Prior to each experiment, the viability of cells was verified by Trypan blue exclusion and only cultures with ≥95% viability were used for experiments. The SkBr3 cell line was originally derived from the pleural effusion of breast adenocarcinoma patient and is known to over-expresses HER-2 (human epidermal growth factor receptor) gene product. Overexpression of this oncogene has been shown to play an important role in the development and progression of certain aggressive types of breast cancer (Moasser, M. M. Oncogene, 26: 6469-6487 (2007)).

SDS-PAGE and Western Blot Analysis of Membrane and Intracellular C1q

Both whole cell and membrane lysates were made using standard procedures. For whole cell lysates, SkBr3 cells were cultured to confluency as described above and were surface labelled with NHS-LLC biotinylation agent and lysed according to the protocol provided by the manufacturer. Briefly, cells were washed 2× with TBS, and re-suspended in 1 ml of lysis buffer. Cells were centrifuged again at 10,000 g at 4° C. after which 100 μl of Neutravidin resin were added to the supernatant and incubated for 1 hr at room temperature (RT). The lysate-resin mixture was flowed through a column and washed 3× with wash buffer. Protein was eluted from resin with DTT and Laemmli buffer. Analysis on SDS-PAGE was performed on mini-PROTEAN precast TGX 10% acrylamide gels with samples being run reduced and alkylated by boiling for 5 min in the presence of 10% 2-β-Mercaptoethanol. After electrophoresis, the protein was electrotransferred to polyvinyl difluoride (PVDF) nitrocellulose membranes, and blocked with 2% BSA in TBST (20 mM Tris-HCl, 150 mM NaCl and 0.05% Tween 20). The bound proteins were then probed with an appropriate dilution of target-specific antibodies, and then visualized by chemiluminescence horseradish peroxidase (HRP)-conjugated species-specific antibody followed by reaction with 4-chloro-1-naphthol substrate.

Detection of Secreted gC1qR (sgC1qR) by Ag-Capture ELISA Assay

SkBr3 cells were seeded in RPMI containing 10% FBS overnight, after which medium was replaced with serum-free RPMI. Cell supernatant was collected after 24, 48, and 72 hr incubations and centrifuged to remove remaining cells. Microtiter wells were coated with 100 μl of 10 μg/ml rabbit anti-gC1qR in carbonate buffer, pH 9.6 (15 mM Na₂CO₃ and 35 mM NaHCO₃) overnight at 4° C. after which the unbound proteins were discarded, and the unreacted sites of the well blocked with 1% BSA (37° C., 1 hr). Next, the wells were washed three times with TBST, and 100 μl of SkBr3 supernatant was added to each well in duplicates and incubated overnight at 4° C. After removal of the unbound proteins, the wells were washed three times with TBST, and bound proteins were detected with biotinylated mAb 60.11 against human gC1qR. The bound gC1qR was then visualized by sequential incubation (37° C., 1 hr each) with Alkaline Phosphatase conjugated streptavidin followed by pNPP solution.

Immunofluorescence Microscopy

Immunofluorescence studies were performed on non-permeabilized SkBr3 cells grown on glass cover slips to 70% confluency. The attached monolayer of cells was then incubated first with PBS containing 0.1% BSA and 1% heat-inactivated human serum or 1 μg/ml Fc fragments to block Fc receptors, followed by incubation with rabbit anti-gC1qR recognizing peptides 50-63, or peptides 144-155, goat anti-C1q or mAb anti-C1q at concentrations of 2.5 pg/ml. After fixing for 10 min with 10% (v/v) formalin, the cover slips were air-dried, placed face down onto microscope slides, sealed using mounting solution (Immu-Mount), and examined by three-dimensional imaging using deconvolution microscopy.

Proliferation Assays

SkBr3 cells were seeded in tissue culture treated 6-well plates at a concentration of 1.25×10⁵ cells/ml in 2 ml of RPMI supplemented with 10% FBS for 96 hrs. The cells were then treated with 10 or 5 μg/ml of purified protein or specific antibodies in duplicates. Untreated or mock-treated cells were used as control. At 96 hr, the supernatant was removed, and the cells were trypsinized, re-suspended in 1 ml of medium and 100 μl of trypan blue was added as an indicator of viability and the cells examined and counted using a hemocytometer. Proliferation studies were conducted separately on plates that were incubated for 48, 72, and 96 hrs. Alternatively, proliferation studies were conducted in which untreated 12-well plates were first coated with 20 μg/ml of either C1q, gC1qR, or various antibodies to C1q in carbonate buffer, pH 9.6 (15 mM Na₂CO₃ and 35 mM NaHCO₃). Control wells were coated with carbonate buffer alone. All wells were coated overnight at room temperature under sterile conditions and subsequently washed with PBS to remove excess buffer. Cells were then seeded at a concentration of 1.25×10⁵ cells/ml in a total volume of 1 ml/well. At the end of the incubation, images were then taken using a light microscope under 10×, every 12 hours for 96 hours at which point all bound and unbound cells were collected and counted using a trypan blue exclusion method as described above.

Statistical Analysis

Student t-tests were performed using statistical software (Excel; Microsoft, Redmond, Wash., USA). A value of p=0.05 was considered to be a significant difference. (n—represents separate experiments performed in duplicates)

### Example 1. Expression of gC1qR, C1q, and Other Proteins on the SkBr3 Cell Surface

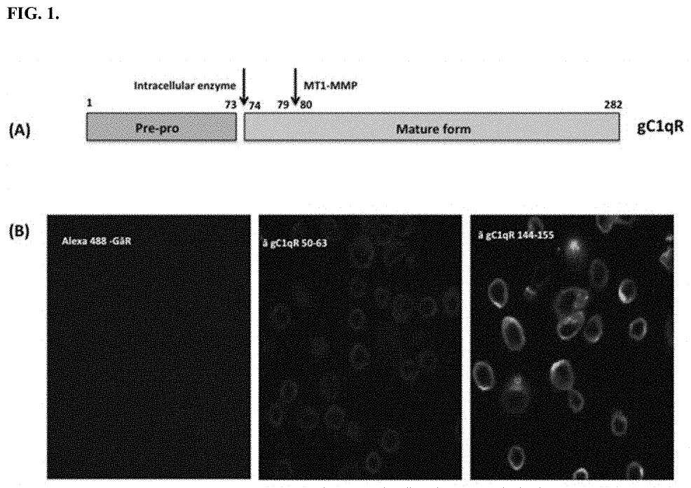

Expression of C1q of gC1qR on SkBr3 cells was examined through a set of deconvolution fluorescence microscopic studies. Previous studies from our laboratory using human umbilical vein endothelial cells (HUVECs) have demonstrated, that the gC1qR molecule is expressed in two forms (FIG. 1A): a “pre-pro” form consisting of residues 1-282 and a “mature” form comprising of residues 74-282, or both (18-19).

To test the expression of the various forms of gC1qR on the SkBr3 cell surface, immunofluorescence studies were performed using two immunoaffinity purified monospecific rabbit anti-gC1qR antibodies: one raised against residues 50-63 within the pre-pro form of gC1qR (region covered by residues 1-73) and another recognizing residues 144-155 gC1qR within the mature form (residues 74-282). Deconvolution fluorescence microscopy was performed on SkBr3 cells grown to confluency on glass cover slips. Cells were incubated with PBS containing 0.1% BSA and 1 mg/ml Fc fragments to block FcRs, followed by incubation with immunoaffinity purified rabbit antigC1qR 144-155. Bound Abs were probed with Alexa Flour 488-anti rabbit Abs.

As shown in FIG. 1B, the staining pattern of these antibodies show higher staining with the Ab recognizing the mature form of gC1qR and detectable, but lower staining, with the Ab which recognizes the pre-pro protein suggesting that the major form of gC1qR expressed on SkBr3 cells is the cleaved or “mature form”. Unlike normal cells, the staining with an anti-gC1qR, which recognizes a specific domain in the mature form of gC1qR (residues 74-282), was much more robust than the minimal staining observed with an antibody, which recognizes a domain in the “pre-pro” region of full-length gC1qR (residues 1-282). Although similar concentration of both antibodies have resulted in very strong staining in normal cultured cells such as HUVECs, indicating the strong presence of both the pre-pro and mature forms of gCqR in non-malignant cells (43), the strong expression in favor of the mature form of gC1qR seen in the SkBr3 line, suggests that most of the “pre-pro” form may have been cleaved by membrane enzymes such as the membrane type 1 MMP (MT1-MMP), which has been shown previously to cleave gC1qR at position Gly⁷⁹-Gln⁸⁰(44), which is close to the N-terminus of the mature or membrane form which starts at Leu⁷⁴.

Another receptor that binds C1q is cC1qR (calreticulin or CR), but requires conditions that are either a low ionic environment or a conformational change in the C1q molecule (25-26). Although others have recently claimed that it also binds to the globular heads (27), under physiological conditions, in our hands cC1qR binds primarily to the collagen tail of C1q, hence the designation cC1qR (25, 28-29). More importantly however, the cC1qR/CR has been shown to form a bi-molecular complex with gC1qR (30) and this protein-protein interaction—especially in the cytoplasm—has been proposed to play a role in anti-apoptotic properties (31).

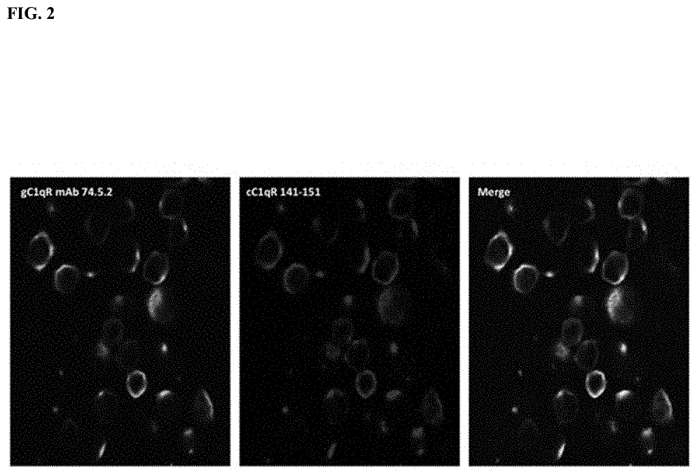

Here we show that the cC1qR/CR is not only abundantly expressed on the SkBr3 cell surface but also is co-localized with gC1qR (FIG. 2). SkBr3 cells grown on glass cover slips were incubated with PBS containing 0.1% BSA and 1 mg/ml Fc fragments to block FcRs. The cells were then probed with mAb 74.5.2 anti-gC1qR and rabbit pAb anti-cC1qR recognizing residues 141-151. Bound Abs were then probed with Alexa Fluor 488-anti-rabbit Abs or Alexa Fluor 594 anti-mouse Abs. Co-localization of gC1qR and cC1qR is shown in the merged picture. Control staining with rabbit non-immune IgG for both experiments showed no fluorescence and was not included.

C1q expression has been documented in various cell types, both epithelial and mesenchymal. Previous reports show the expression of a fibroblast specific C1q, which was then identified in normal intestinal and liver epithelial cells (1). Since SkBr3 cells are epithelial cell-derived cancer cells, we set out to investigate whether they also express C1q by flow cytometry and immunofluorescence studies.

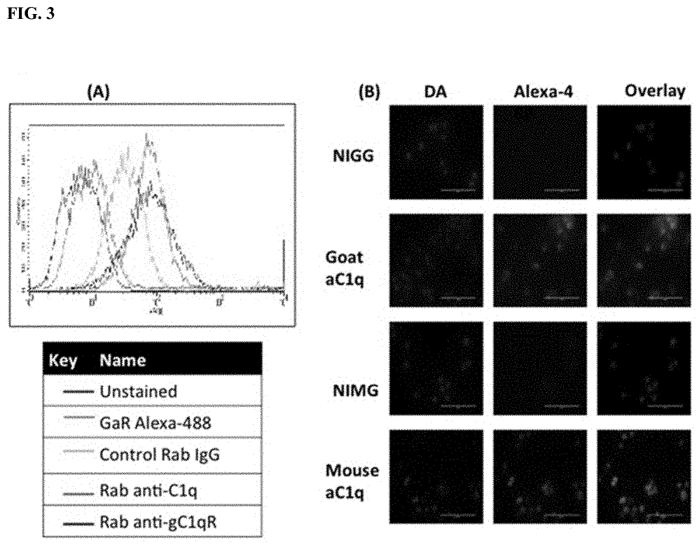

Flow cytometry (FIG. 3A) and immunofluorescence staining (FIG. 3B) using both polyclonal and monoclonal anti-C1q Ab, confirmed the expression of surface C1q, which appears to be similar in intensity to the surface expression of gC1qR (FIG. 3A). As shown in FIG. 3A, flow cytometry analysis was performed on SkBr3 cells grown to confluency and detached using 10 mM EDTA solution. Cells were blocked with Fc fragments for 30 minutes on ice and subsequently probed with either 10 μg of non-immune rabbit IgG (IgG1 Kappa), rab anti-C1q, or rab anti-gC1qR for 30 minutes on ice. Alexa flour 488 conjugated mouse anti-rabbit Ab was incubated for 30 min on ice and cells were then stained with PI to assess viability. As shown in FIG. 3B, immunofluorescence studies on SkBr3 cells grown in chamber slides to 90% confluency were done to determine C1q expression. Monoclonal anti-C1q and goat anti-C1q were used as well as the isotype matched non-immune IgG controls for 30 minutes at room temp. Alexa flour 488 conjugated donkey anti-mouse or rabbit anti-goat were used for 30 minutes at RT after which DAPI stain was applied to visualize the nucleus.

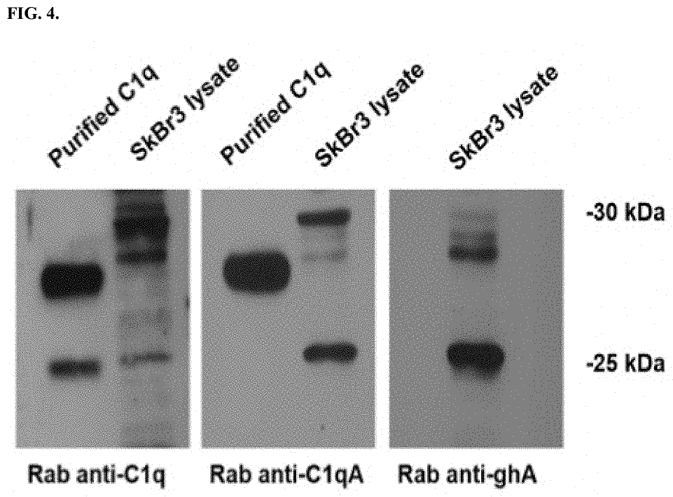

The presence of C1q on SkBr3 cells was demonstrated (see FIGS. 3 and 4). Although some positive staining with the isotype matched non-immune rabbit IgG is also noted, it may be attributed to either the ability of membrane C1q to bind contaminating clusters of IgG or to unblocked Fc receptors. Regardless, the specific staining is significantly higher than the background noise to give confidence.

To verify that the membrane form of C1q has the same structure as plasma C1q, Western-blotting experiments were conducted on SkBr3 cell lysates to assess expression of C1q. Purified C1q (20 ng) was used as a positive control and lysates were probed with isotype matched non-immune rabbit IgG to rule out nonspecific binding of Ab (not shown). Samples were run on 10% polyacrylamide gels and transferred onto nitrocellulose membranes, blocked with 2% BSA in TB ST and incubated overnight at 4° C. with either rabbit anti-aC1q, or rabbit anti-C1q A chain or Rabbit anti-ghA, or NIRG. Rabbit Ab conjugated HRP secondary Ab was used with a chemiluminescence substrate for film exposure.

As shown in FIG. 4, there is a robust presence of C1q in SkBr3 cell lysates. Three distinct antibodies—rabbit anti-C1q, rabbit anti-C1qA, and rabbit anti-ghA—were used to probe 20 μg of whole cell lysates and compared against purified C1q (20 ng). The anti C1q antibody, recognizes a heavier band in the C1q lane at ˜27 kDa band under reducing conditions, and probably includes the B chain, which is camouflaged by the heavy A-chain band—due to its similar molecular weight—and a lower band at approximately 25 kDa, which represents the C chain. The SkBr3 cell derived C1q (lane 2) however, appears to migrate at a slightly higher molecular weight, with the major band (A-chain) migrating at approximately 30 kDa, and other minor bands at 27 and 25 kDa, presumably representing the B and C-chains respectively. As expected, the antibody against the A-chain recognizes the A-band of the purified C1q (lane 3), but also recognizes two bands in the SkBr3 lysate: one at ˜30 and another at ˜25 kDa (lane 4). The lower bands probably represent enzymatic degradation fragments of the heavier A-chain since the same bands are also recognized by highly specific antibody raised against either the A-chain of C1q or to a peptide derived from the globular head region (gh) of the A-chain (lane 5). The fact that the SkBr3 cell lysate-derived C1q runs at a slightly higher molecular weight is not surprising since we anticipated that the membrane-anchored protein would contain an extra membrane-anchoring domain, which is not found in plasma C1q (32).

### Example 2. Both Membrane Expressed cC1qR and gC1qR are Pro-Proliferative Signals

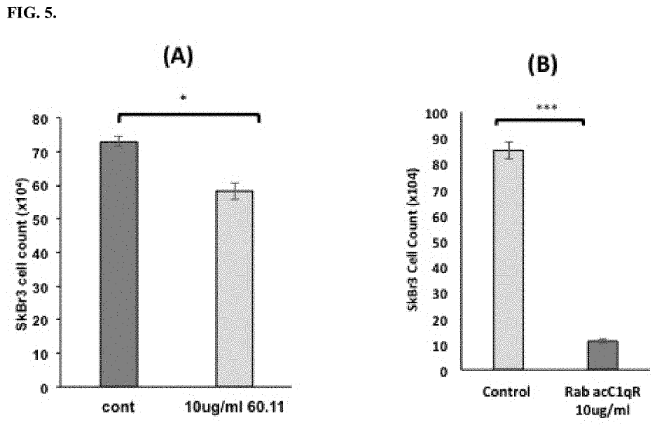

Although a number of cell surface molecules have been claimed to bind to C1q and serve as C1q receptors, (27), we focused here only on the two well-recognized molecules: cC1qR and gC1qR. Since the two receptors, cC1qR and gC1qR, are co-localized on most cells including SkBr3 cells, we compared the relative roles of these receptors in cell proliferation. SkBr3 cells were seeded in 6 well plates at 1×10⁵ cells/ml in the presence or absence of either (A) 10 μg/ml mAb 60.11 recognizing the C1q site on gC1qR or (B) 10 μg/ml affinity-purified rabbit anti-cC1qR recognizing the putative C1q binding domain on cC1qR. After 96 hr, cells were counted in a hemocytometer in the presence of 10 μl/ml trypan blue for cell viability. Results show cell proliferation levels represented as the mean of four assays run in duplicates. Values represent mean±SD of duplicate samples with significance represented by (*p<0.05) and *** (p<0.005) when compared to control samples. As shown in FIG. 5, while blockade with mAb 60.11 resulted in moderate but significant inhibition (*p<0.05), blockade with pAb anti cC1qR-directed against the putative C1q binding domain on cC1qR-resulted in almost complete inhibition of cell proliferation (***p<0.005). This implies that at least on SkBr3 cells, both receptors serve as essential proliferative signals.

### Example 3. The Role of Soluble and Membrane C1q in SkBr3 Cell Culture

Studied were performed to elucidate the relevance of membrane C1q and specifically, the C1q A chain, in cell growth and proliferation. To understand the role of both membrane C1q and gC1qR in SkBr3 cell growth and progression, several co-culture experiments were performed by incubating SkBr3 cells with either C1q or the individual globular head molecules of C1q-ghA, ghB or ghC—to block the cG1qR, or anti-Cq antibodies recognizing either the intact molecule, the C1q A-chain or a region in the ghA that is known to contain a major gC1qR site, to block the membrane C1q. The results show that blockade of C1q with anti-C1q antibodies or blockade of gC1qR with C1q or the C1q gh modules or mAB 60.11 resulted in significant inhibition of cell proliferation.

Proliferation assays were conducted using SkBr3 cells seeded at 1×10⁵ cells/ml in the presence or absence of rabbit anti-C1q (10 μg/ml), anti C1qA (10 μg/ml), or rabbit anti-ghA. Cells were then incubated for 96 hr, after which they were counted in a hemocytometer in the presence of trypan blue. Control wells were either untreated or supplemented with isotype-matched non-immune rabbit IgG (NIRG). Results for NIRG supplemented cells were no different from untreated cells (control) and are not included here. Results are representative of four different experiments run in duplicates. Significance is represented by (** p<0.01) and *** (p<0.005) when compared to control using student's t-test.

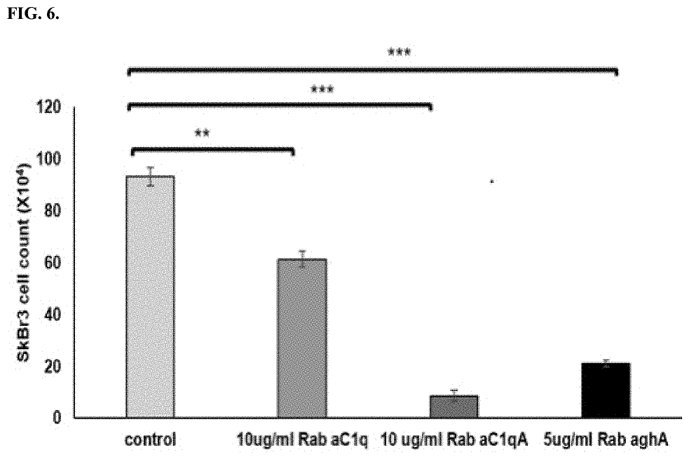

As shown in FIG. 6, while blockade of C1q with anti-C1q resulted in a significant decrease in cell number, blockade of the A-chain with a specific anti C1q-A antibody resulted in a 4-fold decrease in cell number, with more dead cells being observed than live cells. More importantly, blockade with an antibody raised against the gC1qR site on the ghA domain of C1q showed a significant but slightly less cytotoxic effect than the anti-C1q A-chain antibody. These data indicate that the A-chain is an important player in cell proliferation and viability.

To address the relevance of C1q in breast cancer cells, various proliferation assays were performed in which SkBr3 cells were seeded at 1×10⁵ cells/ml in the presence or absence of 10 μg/ml of C1q or its globular head modules, ghA, ghB, or ghC. Proliferation assays were conducted using SkBr3 cells seeded in 6-well plates at 1×10⁵ cells/ml in the presence of either (A) C1q, (B) purified ghA, ghB, or ghC. After 96 hr, Viability was assessed using the trypan blue exclusion assay and viable and non-viable cells counted in a hemocytometer. Results show cell proliferation levels and are represented as the mean of four assays run in duplicates. Significance is represented by (*p<0.05) and *** (p<0.005) when compared to control using student's t-test.

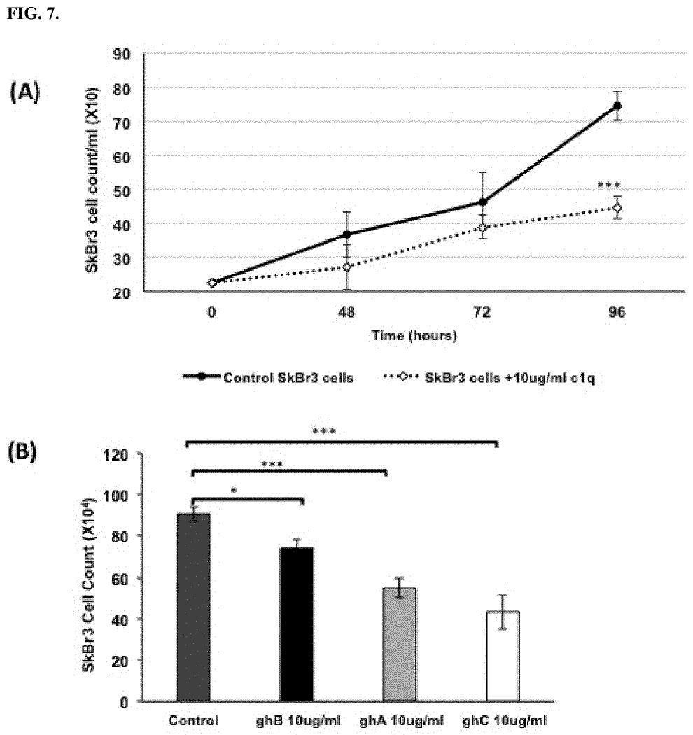

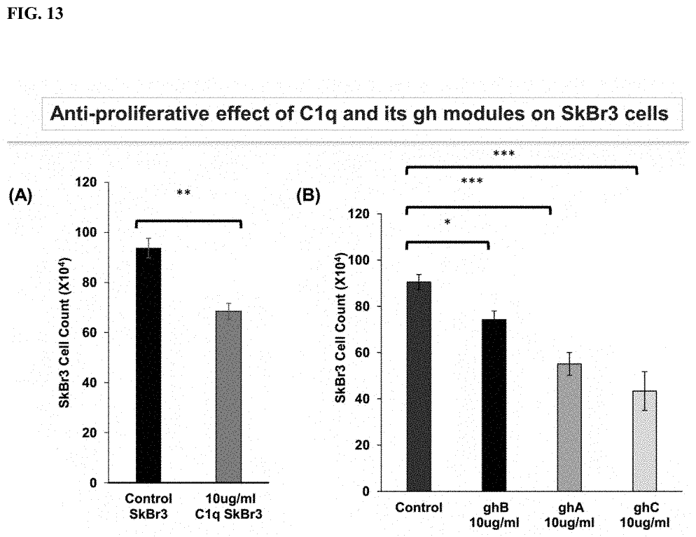

As shown in FIG. 7A, a significant decrease (˜40%) in cell number was observed in C1q treated cells and even a more substantial decrease (70% and 100%) when purified recombinant individual ghA or ghC modules were added to the SkBr3 cells (FIG. 7B, see also FIG. 13). In comparison, the ghB module showed only a modest anti-proliferative effect.

### Example 4: The Role of gC1qR in the Tumor Cell Microenvironment

As shown above, blockade of membrane C1q with antibody results in an anti-proliferative response. Addition of exogenous C1q also results in a strong antiproliferative response presumably by binding primarily to the surface expressed trimeric gC1qR, which can bind efficiently three globular heads at the same time. However, although C1q is abundant in plasma and presumably would venture into the tumor cell microenvironment, it does not seem to reach the tumor cell surface to inflict its antiproliferative potential. Thus, experiments were performed to determine what molecular entities in the tumor cell microenvironment prevent C1q from accessing the tumor cell surface. Since soluble gC1qR can bind C1q with high affinity (14), and tumor cells by and large, secrete soluble gC1qR into the pericellular milieu, we hypothesized that this secreted gC1qR may serve as a molecular shield, which binds C1q in the microenvironment before reaching the cell surface. To test this hypothesis, we first verified that Skbr3 cells release gC1qR into the supernatant using an antigen-capture ELISA. SkBr3 cells were first grown in serum-free medium and supernatants were collected at 24, 48, and 72 hr and tested for the presence of gC1qR.

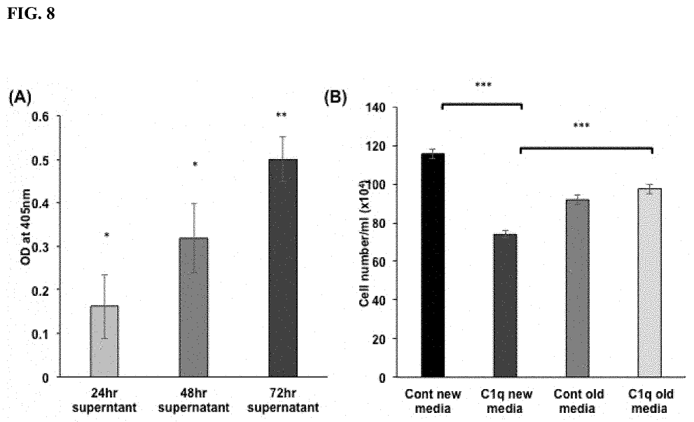

FIG. 8A shows antigen-capture-ELISA on SkBr3 cell supernatants that were taken from cells grown in serum-free RPMI for 24, 48, or 72 hrs (n=4). Microtiter wells were first coated with 100 ul (10 ug/ml) of rabbit anti-gC1qR 144-155 (1 h, 37° C.), blocked with 1% BSA (1 h, 37° C.). Next, 100 μl of either fresh medium or culture supernatants was added and further incubated (1 h, 37° C.). gC1qR was then detected by incubation (1 h, 37° C.) with biotinylated mAb 60.11 (2 ug/ml), AP-neutravidin and PNPP. FIG. 8B shows cell proliferation assay was conducted to assess if gC1qR released by SkBr3 cells in culture after 48 hrs could reverse the anti-proliferative effect of exogenously added C1q. Cells were seeded at 1×10⁵ cells/ml and grown for 48 hr to allow for gC1qR secretion (n=2). Then half the wells had the medium removed to remove residual gC1qR before new medium was added. The other half of the wells kept the old gC1qR-rich medium. Then C1q was added to both wells and incubated for 96 hrs. After incubation, the cells in each well were counted and viability assessed using the trypan blue exclusion assay. Significance is represented by (** p<0.01) and *** (p<0.005) when compared to control using student's t-test.

As shown in FIG. 8A, there was significant and time-dependent secretion of gC1qR. To examine if the secreted gC1qR can block the anti-proliferative effects of exogenously added C1q, SKBr3 cells were first cultured for 48 h to allow measurable secretion of gC1qR, then, the medium was removed from half of the duplicate wells followed by a washing step to remove any residual secreted gC1qR and addition of fresh medium. The other halves of the duplicate wells, on the other hand, were left in the “gC1qR-rich” medium. Subsequently, C1q (10 μg/ml) was added to both cells, and grown for additional 96 hrs. FIG. 8B, shows that while there is a marked decrease in cell number in the wells that had fresh medium—and therefore no residual gC1qR—there was no decrease in cell number in the wells that had the original gC1qR-rich medium. Thus, cells that were maintained in the gC1qR-rich medium were protected from the anti-proliferative effects of C1q presumably because the C1q was “hijacked” and bound by the secreted gC1qR before it reached the cell surface.

To better understand the role of secreted gC1qR in the tumor microenvironment, SkBr3 cells were cultured in the presence of 5 μg/ml and 10 μg/ml gC1qR for 96 hr and cells counted in the presence of trypan blue.

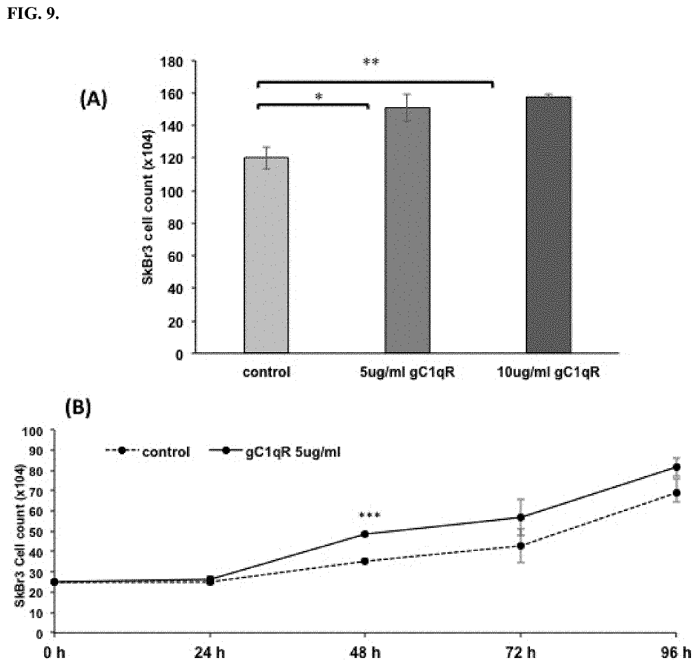

Soluble gC1qR was shown to be an autocrine signal of cell proliferation. Proliferation assay was done in which SkBr3 at 10⁵/ml were co-cultured with gC1qR at 5 μg/ml or 10 μg/ml (n=2) (see FIG. 9A). After 96 hr, cells were trypsinized and counted to determine cell number and viability. Cells were visualized and counted at different time points to determine the time-dependent effect of gC1qR on cell number and viability. (see FIG. 9B). Values represent mean±SD of duplicate samples with significance of (* p<0.05), (** p<0.01), and (***p<0.005), when compared to untreated controls.

As shown in FIG. 9A there was a 30% increase in cell number in gC1qR treated wells with normal viability (≥95%). Interestingly, doubling the concentration of gC1qR did not result in a significant change. Additional proliferation studies were done using the same concentration of gC1qR to determine where in the cell growth curve the effect of gC1qR could first be observed. There was a time dependent increase in cell number at 48 hr, after which no further increase in cell growth could be observed. (FIG. 9B).

### Example 5: Differential Response to Growth by SkBr3 Cells on C1q and gC1qR-Coated Surface

We have shown previously that C1q-coated plates can support and facilitate human umbilical vein derived endothelial cell (HUVEC) adhesion and spreading in a manner that was similar to collagen-coated plates. The C1q-mediated endothelial cell adhesion and spreading in turn was mediated in part by the cooperation of C1q receptors and β1 and α5 integrins (33). Studied were performed to determine if C1q-coated plates would also support SkBr3 cell growth similar to that seen with HUVECS or adversely affect their growth similar to that seen with exogenously added C1q.

Adhesion studies were performed using microplate wells coated with either 20 μg/ml of purified C1q or gC1qR in endotoxin-free carbonate buffer. The cells were then grown for 96 hours and images (4×) taken every 24 hr with a light microscope to show the nature and cell density. Microplate wells coated with either carbonate buffer or tissue culture medium were used as controls.

Microtiter plates (12-well) were coated (overnight at 20° C. under sterile conditions) with either 20 μg/ml of C1q or gC1qR in carbonate buffer, pH 9.6 (15 mM Na₂CO₃ and 35 mM NaHCO₃). Excess buffer was removed and washed with sterile PBS before the addition of 1×10⁵ cells/well in 1 ml of RPMI medium. A light microscope was used to take 4× images of the wells every 24 hours for 96 hours. Buffer coated wells were used as control and did not show any significant difference to cells in culture medium-coated wells. Cell supernatants were collected after 96 hr, and the remaining adherent cells were trypsinized after which supernatants and trypsinized cells were combined, stained with trypan blue and viability established. Students t-tests were applied to determine significance.

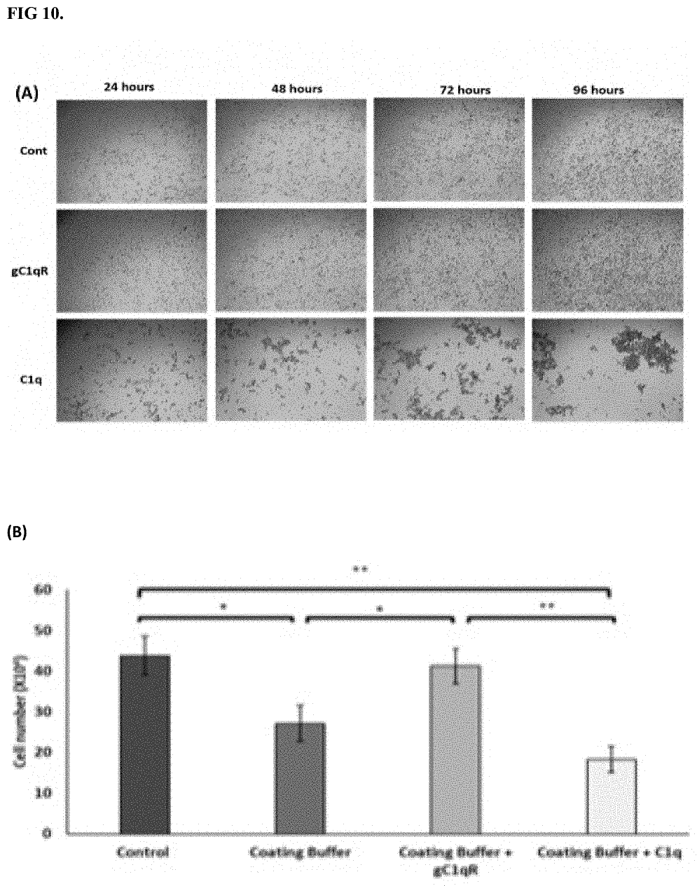

As shown in FIG. 10A, the cells grew at a faster rate with the gC1qR-coated wells and even showed slightly higher confluence than the cells grown in normal tissue culture treated wells. In contrast, cells did not attach and even looked shriveled on the C1q-coated wells. Instead, they formed large clusters that got progressively bigger as the exposure time progressed. After 96 hr, cells were trypsinized, resuspended in fresh medium and analyzed for cell viability using the trypan blue exclusion assay. As shown in FIG. 10B, while cell proliferation was enhanced in gC1qR-coated wells, the opposite response was observed when grown on a C1q-coated surface. Importantly, the gC1qR-coated surface showed about a 40% increase in cell number even when compared to those grown in buffer- or culture medium-coated plates indicating that gC1qR provides a strong growth signal. The cells grown on C1q-coated plates on the other hand exhibited a 50% decrease when compared to cells grown on culture medium-treated plates and a 40% decrease when compared to the cells grown on the coating buffer-coated surface.

### Example 6: ELISA Shows Interaction of ghA Mutants with gC1qR

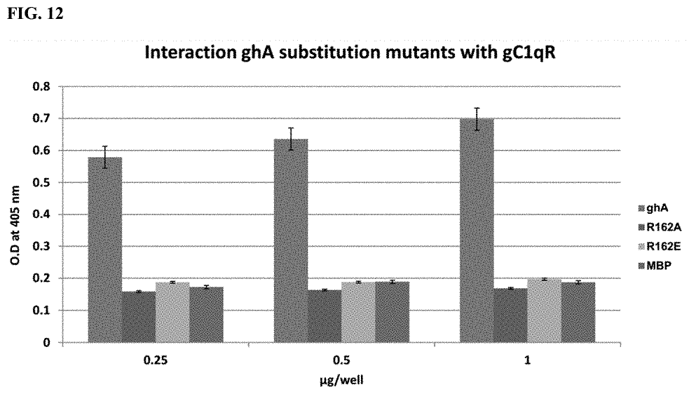

Microtitre wells were coated with 1 μg/well of gC1qR in carbonate buffer and double diluted 2 wells down to give a concentration of 0.5, 0.25 μg/well. The plate was incubated at +4 overnight. The next morning, contents were discarded and wells were blocked for 2 hours with 2% BSA at 37 degrees. Following washing with PBS+0.05% Tween, 2.5 ug/well of ghA wild type, R162A, R162E and MBP was added and the plate was incubated for 1.5 hours at 37 degrees and 1.5 hours at +4. Wells were washed and anti-MBP (1/5000) was added and incubated for 1 hour. Bound protein was detected using IgG-HRP and colour was developed using OPD buffer. The plate was read at a wavelength of 450 nm. The results are shown in FIG. 12.

### REFERENCES

The following references, to the extent that they provide exemplary procedural or other details supplementary to those set forth herein, are specifically incorporated herein by reference.
(1) Morris, K. M., Colten, H. R., and Bing, D. H. (1978). The first component of complement. A quantitative comparison of its biosynthesis in culture by human epithelial and mesenchymal cells. J Exp Med 148(4), 10071019.
(2) Dembitzer, F. R., Kinoshita, Y., Burstein, D., Phelps, R. G., Beasley, M. B., Garcia, R., et al. (2012). gC1qR expression in normal and pathologic human tissues: differential expression in tissues of epithelial and mesenchymal origin. J Histochem Cytochem 60(6), 467-474. doi: 10.1369/0022155412440882.
(3) Peerschke, E. I., and Ghebrehiwet, B. 2014. cC1qR/CR and gC1qR/p33: observations in cancer. Mol Immunol 61: 100-109.
(4) Bossi, F., Tripodo, C., Rizzi, L., Bulla, R., Agostinis, C., Guarnotta, C., et al. (2014). C1q as a unique player in angiogenesis with therapeutic implication in wound healing. Proc Natl Acad Sci USA 111(11), 4209-4214. doi: 10.1073/pnas.1311968111.
(5) Kim, B. C., Hwang, H. J., An, H. T., Lee, H., Park, J. S., Hong, J., et al. (2016). Antibody neutralization of cell-surface gC1qR/HABP1/SF2-p32 prevents lamellipodia formation and tumorigenesis. Oncotarget 7(31), 4997249985. doi: 10.18632/oncotarget.10267.
(6) Kim, K. B., Yi, J. S., Nguyen, N., Lee, J. H., Kwon, Y. C., Ahn, B. Y., et al. (2011). Cell-surface receptor for complement component C1q (gC1qR) is a key regulator for lamellipodia formation and cancer metastasis. J Biol Chem 286(26), 23093-23101. doi: 10.1074/jbc.M111.233304.
(7) Kaur, A., Sultan, S. H. Murugaiah, V., Pathan, A. A., Alhamlan, F. S., Karteris, E. and Kishore, U. 2016. Human C1q induces apoptosis in an ovarian cancer cell line via tumor necrosis factor pathway. Front Immunol 7: 599. doi: 10.3389/fimmu.2016.00599
(8) Bulla, R., Tripodo, C., Rami, D., Ling, G. S., Agostinis, C., Guarnotta, C., et al., 2016. C1q acts in the tumour microenvironment as a cancer-promoting factor independently of complement activation. Nat Commun 7: 10346.
(9) Ghebrehiwet, B., Hosszu, K., Valentino, A., and Peerschke, E. I. (2012). The C1q family of proteins: insights into the emerging non-traditional functions. Front Immunol 3. doi: 10.3389/fimmu.2012.00052.
(10) Ghebrehiwet, B., Hosszu, K., and Peerschke, E. I. 2016. C1q as an autocrine and paracrine regulator of cellular functions. Mol Immunol. 84:26-33
(11) Ghebrehiwet, B., Habicht, G. S., and Beck, G. 1990. Interaction of C1q with its receptor on cultured cell lines induces an anti-proliferative response. Clin Immunol Immunopathol 54: 148-160.
(12) Habicht, G. S., Beck, G., and B. Ghebrehiwet. 1987. C1q inhibits the expression of B lymphoblastoid cell line interleukin 1 (IL 1). J Immunol 138: 2593-2597.
(13) Chen, A., Gaddipati, S., Hong, Y., Volkman, D. J., D. J., Peerschke, E. I. and Ghebrehiwet, B. 1994. Human T cells express specific binding sites for C1q. Role in T cell activation and proliferation. J Immunol 153: 1430-1440.
(14) Tacnet, P., Cheong, E. C. C., Goeltz, P., Ghebrehiwet, B., Arlaud, G. J., Liu, X-Y., et al. 2008. Trimeric reassembly of the globular domain of human C1q. BBA-Proteins and Proteomics. 1784: 518-529.
(15) Hong, Q., Sze, C. I., Lin, S. R., Lee, M. H., He, R. Y., Schultz, et al. 2009. Complement C1q activates tumor suppressor WWOX to induce apoptosis in prostate cancer cells. PLoS One 4: e5755.
(16) Miyamae, Y., Mochizuki, N. S., Shimoda, M., Ohara, K., Abe, H., Yamashita, S., et al., 2016. ADAM28 is expressed by epithelial cells in human normal tissues and protects from C1q-induced cell death. FEBS J 283: 1574-1594.
(17) Peerschke, E. I., Brandwijk, R. J., Dembitzer, F. R., Kinoshita, Y., and Ghebrehiwet, B. 2015. Soluble gC1qR in Blood and Body Fluids: Examination in a Pancreatic Cancer Patient Cohort. Int. J Cancer Res. Mol. Mech. 1. 2015 October; 1(3): 10.16966/ijcrmm.110.
(18) Ghebrehiwet, B., Lim, B. L., Peerschke, E. I., Willis, A. C., and K. B. Reid. 1994. Isolation, cDNA cloning, and overexpression of a 33-kD cell surface glycoprotein that binds to the globular “heads” of C1q. J Exp Med 179: 1809-1821.
(19) Ghebrehiwet, B., Jesty, J., and Peerschke, E. I. 2002. gC1q-R/p33: structure-function predictions from the crystal structure. Immunobiology 205: 421-432.
(20) Lim, B. L., Reid, K. B., Ghebrehiwet, B., Peerschke, E. I., Leigh, L. A., and K. T. Preissner. 1996. The binding protein for globular heads of complement C1q, gC1qR. Functional expression and characterization as a novel vitronectin binding factor. J Biol Chem 271: 26739-26744.
(21) Kojouharova, M. S., Gadjeva, M. G., Tsacheva, I. G., Zlatarova, A., Roumenina, L. T., Tchorbadjieva, M. I., et al., 2004. Mutational analyses of the recombinant globular regions of human C1q A, B, and C chains suggest an essential role for arginine and histidine residues in the C1q-IgG interaction. J Immunol 172: 4351-4358.
(22) Kishore, U., Gupta, S. K., Perdikoulis, M. V., Kojouharova, M. S., Urban, B. C., and Reid. K. B. 2003. Modular organization of the carboxyl-terminal, globular head region of human C1q A, B, and C chains. J Immunol 171: 812-820.
(23) Ghebrehiwet, B., Lu, P. D., Zhang, W., Lim, B-L, Eggleton, P., Leigh, L. E. A., et al. 2009. Identification of functional domains on gC1q-R, a cell surface protein that binds to the globular “heads” of C1q, using monoclonal antibodies and synthetic peptides. Hybridoma. 15: 333-342.
(24) Moasser, M. M. 2007. “The oncogene HER2: its signaling and transforming functions and its role in human cancerpathogenesis. Oncogene, 26: 6469-6487.
(25) Malhotra, R., Willis, A. C., Jensenius, J. C., Jackson, J., Sim, R. B., 1993. Structure and homology of human C1q receptor (collectin receptor). Immunology 78, 341-348.
(26) Steinø, A., Jøtgensen, C. S., Lursen, I., and Houen, G. 2004. Interaction of C1q with the receptor calreticulin requires conformational change in C1q. Scand J Immunol 59:485-495.
(27) Thielens, N. M., Tedesco, F., Bohlson, S. S., Gaboriaud, C., and Tenner, A. J. 2017. C1q: A fresh look at an old molecule. Mol. Immunol. 89:73-83.
(28) Ghebrehiwet, B., Silvestri, L., McDevitt, C. 1984. Identification of the Raji cell membrane-derived C1q inhibitor as a receptor for human C1q. Purification and characterization. J Exp. Med. 160: 1375-1389.
(29) Eggleton, P., Lieu, T. S., Zappi, E. G., Sastry, K. Coburn, J., Zaner, K. S., et al., 1994. Calreticu-lin is released from activated neutrophils and binds to C1q and mannan-binding protein. Clin. Immunol. Immunopathol. 72, 405-409.
(30) Ghebrehiwet, B., Lu, P. D., Zhang, W., Keilbaugh, S. A., Leigh, L. E., Eggleton, P., et al. 1997. Evidence that the two C1q binding membrane proteins, gC1q-R and cC1q-R, associate to form a complex. J Immunol 159: 1429-1436.
(31) Watthanasurorot, A., Jiravanichpaisal, P., Soderhall, K., and Soderhall, I. 2013. A calreticu-lin/gC1qR complex prevents cells from dying: a conserved mechanism from arthropods to humans. J Mol Cell Biol 5: 120-131.
(32) Trinder, P. K., Maeurer, M. J., Kaul, M., Petry, F. and Loos, M. 1993. Functional domains of the human C1q A-chain. Behring Inst Mitt: 93:180-188.
(33) Feng, X., Tonnesen, M. G., Peerschke, E. I., and Ghebrehiwet, B. 2002. Cooperation of C1q re-ceptors and integrins in C1q-mediated endothelial cell adhesion and spreading. J Immu-nol 168: 2441-2448.
(34) White, T. K., Zhu, Q., and Tanzer, M. L. 1995. Cell surface calreticulin is a putative mannoside lectin, which triggers mouse melanoma cell spreading. J Biol Chem, 270: 15926-15929.
(35) Ghosh I., Chowdhury, A. R., Rajeswari, M. R., and Datta, K. 2004. Differential expression of hy-aluronic acid binding protein 1 (HABP1)/P32/C1QBP during progression of epidermal carcinoma. 2004. Mol Cell Biochem. 2004: 267(1-2):133-139.
(36) Rubinstein D. B., Stortchevoi, A., Boosalis, M., Ashfaq, R., Ghebrehiwet, B., Peerschke, E. I., et al. 2004. Receptor for the globular heads of C1q (gC1qR, p33, hyaluronan binding protein), is preferentially expressed by adenocarcinoma cells. Int J Cancer. 110: 741-750.
(37) Chen, Y. B., Jiang, C. T., Zhang, G. Q., Wang, J. S., and Pang, D. 2009. Increased expression of hyaluronic acid binding protein 1 is correlated with poor prognosis in patients with breast cancer. J Surg Oncol. 100: 382-386.
(38) Fogal V., Richardson, A. D., Karmali, P. P., Scheffler, I. E., Smith, J. W., and Ruoslahti E. 2010. Mitochondrial p32 protein is a critical regulator of tumor metabolism via maintenance of oxidative phosphorylation. Mol. Cell Biol. 30:1303-1318.
(39) McGee, A., Douglas, D. L., Liang, Y., Hyder, S. M., and Baines, C. P. 2011. Thw mitochondrial protein C1qbp promotes cell proliferation, migration and resistance to cell death. Cell Cycle. 10: 4119-4127.
(40) Wang J., Song, Y., Liu, T., Shi, Q., Zhong, Z., Wei, et al. 2015. Elevated expression of HABP1 is a novel prognostic indicator in triple-negative breast cancers. Tumour Biol. 36: 4793-4799.
(41) Winslow, S., Lenaderson, K., and Edsjö, A. 2015. Prognostic stromal gene signatures in breast cancer. Breast Cancer Res. 17:1-31.
(42) Jiang, Y., Wu, H., Liu, J., Chen, Y., Xie, J., Zhao, Y., and Pang, D. 2017. Increased breast can-cer risk with HABP1/p32/gC1qR genetic polymorphism rs2285747 and its upregulation in northern Chinese women. Oncotarget, 8:13932-13941.
(43) Ghebrehiwet, B., Ji, Y., Valentino, A., Pednekar, L., Ramadass, M., Habiel, D., et al. 2014. Sol-uble gC1qR is an autocrine signal that induces B1R expression on endothelial cells. J Immunol 192: 377-384.
(44) Rozanov, D. V., Ghebrehiwet, B., Ratnikov, B., Monosov, E. Z., Deryugina, E. I., and Strongin, A. Y. 2002. The cytoplasmic tail peptide sequence of membrane type-1 matrix metallo-proteinase (MT1-MMP) directly binds to gC1qR, a compartment-specific chaperone-like regulatory protein. FEBS Lett 527:51-57.
(45) Michalak, M., Corbett, E. F., Mesaeli, N., Nakamura, K., and Opas, M. 1999. Calreticulin: one protein, one gene, many functions. Biochemical Journal, vol. 344: 281-292.
(46) van Leeuwen H. C. and P. O'Hare. 2001. Retargeting of the mitochondrial protein p32/gC1qR to a cytoplasmic compartment and the cell surface. J Cell Sci. 114:2115-2123.
(47) Hosszu, K. Valentino, A., Vinayagasundaram, U., Vinayagasundaram, R., Joyce, M. G., Ji, Y., et al. 2012. DC-SIGN, C1q and gC1qR form a trimolecular receptor complex on the sur-face of monocyte-derived dendritic cells. Blood. 120:1228-1236.
(48) Xu, Z., Hirasawa, A., Shinoura, H., and Tsujimoto G. 1999. Interaction of the alpha (1B)-adrenergic receptor with gC1q-R, a multifunctional protein. J Biol Chem. 27:21149-21154.
(49) Ghirian, I., Klickstein, L. B. and Nicholson-Weller, A. 2003. Calreticulin is at the surface of cir-culating neutrophils and uses CD59 as an adaptor molecule. J Biol Chem 279:21024-21031.
(50) Kaul, M, and Loos, M. 1995. Collagen-like complement C1q is a membrane protein of human monocyte-derived macrophages that mediates endocytosis. J Immunol. 155:5795-5802.
(51) Pednekar, L, Valentino, A., Ji, Y., Tumma, N., Valentino, C., Hosszu, K., et al. 2016. Identifica-tion of the gC1qR sites for the HIV-1 viral envelope protein gp41 and HCV core protein: Implications in viral-specific pathogenesis and therapy. Mol Immunol. 74:18-26.
(52) Jiang H., Rummage, J. A., Stewart, C. A., Herriott, M. J., Kolosova, I., Kolosov, M., et al. 1996. Evidence for endogenous C1q modulates TNF-alpha receptor synthesis and autocrine binding of TNF-alpha associated with lipid A activation of murine macrophages for nitric oxide production. Cell Immunol. 170:34-40.
(53) Van den Berg, R. H., Feber-Krol, M. C., Sim, R. B. and Daha, M. R. 1998. The first subcomponent of complement C1q triggers the production of IL-8, Il-6, and monocyte chemoattractant peptide-1 by human umbilical vein endothelial cells. J. Immunol. 161:6924-6930.
(54) Fuchs Y., Steller, H. 2015. Live to die another way: modes of programmed cell death and the signals emanating from dying cells. Nat Rev Mol Cell Biol. 16: 329-344.
(55) Shapiro, L., and Scherer, P. E. 1998. The crystal structure of a complement-1q family of protein suggests an evolutionary link to tumor necrosis factor. Curr. Biol. 8, 335-338.
(56) Kishore, U., Gaboriaud, C., Waters, P., Shrive, A. K., Greenhough, T. J., Reid, K. B. et al. 2004. C1q and tumor necrosis factor superfamily: modularity and versatility. Trends Immunol 25: 551-561.
(57) Kittlesen, D. J., Chianese-Bullock, K. A., Yao, Z. Q., Braciale, T. J., and Hahn, Y. S. 2000. Interaction between complement receptor gC1q-R and hepatitis C virus core protein inhibits T-lymphocyte proliferation. J Clin Invest. 106:1239-1249.
(58) Cummings, K. L, Rosen, H. R., and Hahn, Y. S. 2009. Frequency of gC1qR⁺CD4⁺ T cells increases during acute Hepatitis C virus infection and remains elevated in patients with chronic infection. Clin Immunol. 132:401-411.
(59) Choi, Y., Kwon, Y. C., Kim, S. I., Park, J. M., Lee, K. H., and Ahn, B. Y. 2009. A hantavirus causing hemorrhagic fever with renal syndrome requires gC1qR/p32 for efficient cell binding and infection. Virology. 381:178-183.
(60) Fausther-Bonvendo, H., Vieillard, V., Sagan, S., Bismuth, G., and P. Debre 2010. HIV gp41 engages gC1qR on CD4⁺ T cells to induce the expression of an NK ligand through the PIP3/H2O2 pathway. PLoS Pathog. 2010:e1000975.
(61) Biswas, A. K., Hafiz, A. Benerjee, B., Kim, K. S., K. S., Datta, K., and Chitnis, C. E. 2007. Plasmodium falciparum uses gC1qR/HABP/p32 as a receptor to bind to vascular endothelium and platelet-mediated clumping. PLOS Pathogens. 3(9): e130.
(62) Peterson, K., Zhang, L, W., Lu, P. D., Keilbaugh, S. A., Peerschke, E. I. and Ghebrehiwet, B. 1997. The C1q-binding cell membrane proteins cC1q-R and gC1q-R are released from activated cells: subcellular distribution and immunochemical characterization. Clin Immunol Immunopathol 84: 17-26.
(63) Phagoo, S. B., Reddi, K., Anderson, K. D., Leeb-Lundberg, L. M., and Warburton, D. 2001. Brady-kinin B1 receptor up-regulation by interleukin-1 and B1 agonist occurs through inde-pendent and synergistic intracellular signaling mechanisms in human lung fibroblasts. J. Pharmacol. Exp. Ther., 298: 77-85.
(64) Guo, Y. L. and Colman, R. W. 2005. Two faces of high-molecular-weight kininogen (HK) in an-giogenesis: bradykinin turns it on and cleaved HK (HKa) turns it off. J Thomb Haemost 3:670-676.
(65) Ghebrehiwet, B., Kaplan, A. P., Joseph, K. and Peerschke, E. I. 2016. The complement and con-tact activation systems: partnership in pathogenesis beyond angioedema. Immunol Rev. 274: 281-289.
(66) Kaplan, A. P. and Ghebrehiwet, B. 2010. The plasma bradykinin-forming pathways and its inter-relationships with complement. Mol Immunol 47:2161-2169.
(67) Schmaier, A. H. 2016. The contact activation and kallikrein/kinin systems: pathophysiologic and physiologic activities. J Thromb Haemost 14:28-39
(68) Guo, R, Fredrik Leeb-Lundberg, L. M., Madden, J. F., and Daaka, Y. 2003. Receptor subtype 1 expression and function in prostate cancer. Cancer Res 63:2037-2041.
(69) Leigh. L. E., Ghebrehiwet, B., Perera, T. P., Bird, I. N., Strong, P., Kishore, U., Reid, K. B., and Eggleton, P. 1998. C1q-mediated chemotaxis by human neutrophils: involvement of gC1qR and G-protein signaling mechanisms. Biochem J 330:247-254.72.
(70) Oiki S., and Okada, Y. 1998. C1q induces chemotaxis and K+ conductance activation coupled to increased cytosolic Ca+ in mouse fibroblasts. J Immunol. 141:3177-3185.
(71) Vegh, Z., Kew, R. R., Gruber, B. L., and Ghebrehiwet, B. 2006. Chemotaxis of human mono-cyte-derive dendritic cells to complement C1q is mediated by the receptors gC1qR and cC1qR. Mol Immunol. 43: 1402-1407.
(72) Greco, S., Elia, M. G., Muscella, A., Romano, S., Storelli, C., and Marsigliante, S. 2005. Brad-ykinin stimulates cell proliferation through an extracellular-regulated kinase 1 and 2-dependent mechanism in breast cancer cells in primary culture. J Endocrinol 186: 291-301.
(73) da Costa, P. L., Sirois, P., Tannock, I. F., and Chammas, R. 2014. The role of kinin receptors in cancer and therapeutic opportunities. Cancer Lett 345: 27-38.
(74) Luddington, S., Quanstrom, E. E. Page, R. C. and Bordin, S. 1993. Expression and function of gingival fibroblast C1q receptors are upregulated by Interleukin-13 and transforming growth factor-3. J Cell Phys. 155: 157-163.

We claim:

1. A method of treating cancer in a patient in need thereof comprising administering to the patient a therapeutically effective amount of an agent that is capable of binding to membrane-bound C1q.

2. The method of claim 1, wherein the agent is an antibody.

3. The method of claim 2, wherein the antibody binds to the A-chain of C1q.

4. The method of claim 2, wherein the antibody binds to the C-chain of C1q.

5. The method of claim 3, wherein the antibody binds to the gC1qR binding site on the A-chain of C1q.

6. The method of claim 1, wherein the agent is a gC1qR peptide that blocks binding sites selected from the group consisting of HK and C1q binding sites.

7. The method of claim 1, wherein the agent is an antibody or peptide that blocks binding of the cC1qR receptor to membrane bound C1q.

8. The method of claim 1, wherein the agent is an antibody that blocks the interaction between gC1qR and ghA of the C1q.

9. The method of claim 1, wherein the agent is an antibody that blocks the interaction between gC1qR and ghC of the C1q.

10. The method of claim 1, wherein the agent is administered directly into a tumor mass.

11. The method of claim 1, wherein the inhibitor is administered intravenously.

12. The method of claim 1, further comprising administering to the patient an additional cancer therapy.

13. The method of claim 12, wherein the additional cancer therapy is a monoclonal antibody.

14. The method of claim 12, wherein the additional cancer therapy is a small-molecule based immunotherapy.

15. The method of claim 12, wherein the additional cancer therapy is radiotherapy, chemotherapy, hormonal therapy, immunotherapy, or toxin therapy.

16. The method of claim 1, wherein the cancer is selected from the group consisting of breast cancer, mesothelioma, melanoma, colon cancer and prostate cancer.

17. The method of claim 16, wherein the cancer is breast cancer.

18. A method for inhibiting cell proliferation comprising contacting a cell expressing membrane-bound C1q with an agent that inhibits the interaction of soluble gC1qR with the membrane-bound C1q.

19. The method of claim 18, wherein the agent is an antibody.

20. The method of claim 19, wherein the antibody binds to the membrane-bound C1q.

21. The method of claim 20, wherein the antibody binds to the A-chain of C1q.

22. The method of claim 21, wherein the antibody binds to the gC1qR binding site on the A-chain of C1q.

23. The method of claim 19, wherein the antibody binds to the C-chain of C1q.

24. The method of claim 19, wherein the antibody blocks the interaction between gC1qR and ghA of the C1q.

25. The method of claim 19, wherein the antibody blocks the interaction between gC1qR and ghC of the C1q.
